SEQUENCE LISTING (2 sequences)

SEQ ID NO: 1 - protein, 31 aa, synthetic construct
HAEGTFTSDVSSYLEGQAAKEFIAWLVRGRG

SEQ ID NO: 2 - protein, 31 aa, synthetic construct
  MOD_RES 2: note = Aib
HXEGTFTSDVSSYLEGQAAKEFIAWLVRGRG

(19) United States
(12) Patent Application Publication
(10) Pub. No.: US 2025/0241994 A1
(43) Pub. Date: Jul. 31, 2025

(54) PHARMACEUTICAL FORMULATION OF GLUCAGON-LIKE PEPTIDE -1 RECEPTOR AGONIST PEPTIDE FOR SUBLINGUAL DELIVERY

(71) Applicant: Immunwork Inc., Taipei (TW)

(72) Inventors: Tse-Wen CHANG, Taipei City (TW); Chien-Jen CHEN, Taipei City (TW); Hsing-Mao CHU, Taipei City (TW)

(21) Appl. No.: 19/026,765

(22) Filed: Jan. 17, 2025

Related U.S. Application Data
(60) Provisional application No. 63/626,512, filed on Jan. 29, 2024.

(51) Int. Cl. A61K 38/26; A61K 9/00; A61K 9/20; A61K 9/48; A61K 47/46; A61P 3/04; A61P 3/10

(52) U.S. Cl. CPC A61K 38/26; A61K 9/006; A61K 9/2027; A61K 9/2068; A61K 9/4825; A61K 47/46; A61P 3/04; A61P 3/10

(57) ABSTRACT

Disclosed herein are formulations suitable for sublingual administration in the treatment of obesity, type II diabetes, or disorders related thereto. The formulation includes 5-10 mg of a glucagon-like peptide-1 receptor agonist peptide (GLP-1 RA-P) dissolved in 1.5-2.0 mL of a buffer solution, and a pH value of about 5.5 to 7.5. Also disclosed herein are methods for treating obesity, type II diabetes, or disorders related thereto. The method includes sublingually administering the present formulation to the subject and keeping the formulation under the tongue of the subject for 5 to 10 minutes, wherein the sublingual administration of the formulation achieves 2-10% bioavailability of the GLP-1 RA-P as compared to that achieved by subcutaneous injection.

### REFERENCE TO A SEQUENCE LISTING

The present application is being filed along with a Sequence Listing in electronic format. The Sequence Listing is provided as a file entitled “P4375_SEQ_AF”, created Jan. 10, 2025, which is 4 KB in size. The information in the electronic format of the Sequence Listing is incorporated herein by reference in its entirety.

### CROSS-REFERENCES TO RELATED APPLICATIONS

This application claims priority and the benefit to U.S. Provisional Patent Application No. 63/626,512, filed Jan. 29, 2024, the entirety of which is incorporated herein by reference.

### BACKGROUND OF THE INVENTION

### 1. Field of the Invention

The present disclosure relates to the field of pharmaceutical formulations. More specifically, it relates to pharmaceutical formulations suitable for sublingual administration and their uses in the treatment of overweight or hyperglycemia related disorders.

### 2. Description of Related Art

### Diabetes and Obesity are Major Diseases Confronting Human Populations

Diabetes is an endocrine disease characterized by high blood glucose levels, the presence of glucose in urine, and elevated glycosylated hemoglobin (HbA1c) in the blood. It leads to a broad spectrum of macrovascular complications, including coronary artery disease, stroke, and peripheral artery disease, as well as microvascular diseases affecting the eyes, kidneys, and nerves. Diabetes is commonly manifested with high blood pressure, heart disease, fatty liver disease, and nonalcoholic steatohepatitis (NASH), and chronic ulcers in the legs and feet. Type 1 diabetes, which accounts for 10-15% of all diabetes cases, results from the failure of the pancreas to secrete sufficient insulin and therefore can be treated with insulin. In contrast, type 2 diabetes comprises approximately 80% of all diabetes cases and is characterized by insulin resistance. Treatment of Type 2 diabetes typically involves metformin, GLP-1 receptor agonists, and lifestyle modification, such as exercise and bodyweight management.

In recent years, the medical community and governmental healthcare authorities have increasingly recognized obesity as a major disease rather than merely a lifestyle issue. Defined as a body mass index (BMI) over 30 kg/m2, obesity is linked to a higher risk of type 2 diabetes, cardiovascular diseases, obstructive sleep apnea, osteoarthritis, depression, and certain types of cancer. The prevalence of obesity and diabetes—often not comorbid—has rapidly increased, affecting 10 to 30% of the general population in many countries. These conditions place significant demands on healthcare resources and contribute to substantial economic burdens.

### GLP-1 RAs are Major Pharmaceuticals for Treating Type-2 Diabetes and Obesity

Glucagon-like peptide 1 (GLP-1) is produced and secreted by intestinal enteroendocrine L cells and certain neurons in the brainstem upon food consumption. The initially produced GLP-1 (1-37) undergoes proteolytic cleavage, resulting in the biologically active forms GLP-1 (7-36) amide and GLP-1 (7-37). GLP-1 plays a significant role in regulating various organs, including the pancreas, stomach, liver, muscles, adipose tissue, kidneys, brain, and bones. One of its key functions is to work alongside gastric inhibitory polypeptide (GIP), also known as glucose-dependent insulinotropic peptide, in stimulating insulin secretion and inhibiting glucagon release from the pancreas, which helps lower blood glucose levels. Additionally, GLP-1 inhibits gastric emptying, acid secretion, and motility in the stomach, collectively reducing appetite.

Several peptides act as GLP-1 receptor agonists (referred herein as GLP-1 RA-Ps) have been developed and approved as therapeutic agents for treating type 2 diabetes and obesity. However, they are all administered via subcutaneous injection, except for the oral formulation of semaglutide (Rybelsus). In the large populations affected by type-2 diabetes or obesity, many patients are fearful of needles. An injection-free delivery method could enhance the acceptance and compliance of these treatments, especially among new patients. Promoting self-administration or home use of the medication would further benefit from such a delivery system. Among all injection-free delivery options for therapeutical peptides and proteins, including GLP-1 RA-Ps, Rybelsus represents the status of the art, for the reason that a therapeutically effective amount of semaglutide can be delivered orally with a once per day regimen. However, Rybelsus has a very low bioavailability of about 0.1-1%, and requires optimal conditions for absorption.

### Bioavailability is the Key for the Feasibility of Sublingual Delivery of Peptide Drugs

For peptide and protein drugs, parenteral administration via intravenous, subcutaneous, and intramuscular injections is standard practice. This is because peptides and proteins are digested in the stomach by various digestive enzymes, making oral administration ineffective. Very few peptide drugs have been developed for oral or sublingual delivery, which requires transport across the mucosal epithelial layer.

In general, bioavailability of a drug in an administered subject can be defined as the proportion (or %) of the administered drug that is present in the body of the administered subject over a given observation period. When administered subcutaneously, intradermally, or intramuscularly, these drugs often achieve relatively high bioavailability, sometimes approaching 100%. This is contingent on the extent to which the drug is digested or inactivated in the tissues as it diffuses into the blood circulation from the injection site. The relatively high bioavailability associated with parenteral administration also applies to protein and peptide drugs.

Oral or sublingual administration is highly desirable, as many patients fear needles, and injection can negatively impact compliance. The convenience and comfort of oral and sublingual routes may enhance medication adherence. However, very few protein and peptide drugs are approved for these routes. Notable exceptions include oxytocin and a synthetic version of desmopressin, which can be administered sublingually in addition to injection. Semaglutide (Rybelsus) has also been approved as an oral tablet, designed with a unique formulation and specific timing of food and water intake. Moreover, oral formulations of human growth hormone are currently being tested in human Phase II clinical trials. These proteins or peptides are hormones, functioning at very low concentrations, which facilitate their achieving functional bioavailability.

The sublingual formulation of Oxytocin has bioavailability with a 10-fold variation between 0.007% and 0.07% in treated human subjects (De Groot A N et al, J Pharm Pharmacol (1995) 47:571-5). The absolute bioavailability of sublingually administered desmopressin varies in treated human subjects between 0.21% and 0.31% (Electronic Medicines Compendium. Desmopressin 120 microgram Sublingual Tablets. Molecule Pharma Ltd., Aug. 30, 2023; Oiso Y, et. al. J Clinical Endocrinology & Metabolism (2013) 98 (10), 3958-3967). The oral form of semaglutide (Rybelsus) has bioavailability in treated human subjects of 0.4-1%, and requires optimal conditions for absorption (Novo Nordisk Inc. Rybelsus Prescribing Information, October 2019)

### Need for an Improved Sublingual Delivery for GLP-1 RA-Ps

Sublingual delivery has been developed, marketed and used clinically only for two peptide hormones, oxytocin and desmopressin. In contrast, sublingual delivery methods for other peptide and protein drugs remain undeveloped. Thus, there exists in the related art a need for an improved oral formulation of GLP-1 RA-Ps for treating obesity or type II diabetes, in which a 7-10% bioavailability of GLP-1 RA-Ps is achieved by the administration of the present improved sublingual formulation.

### SUMMARY

The present disclosure is based on the discovery that GLP-1 RA-Ps may be successfully transported across sublingual mucosal membrane. Accordingly, in one aspect, the present disclosure provides a pharmaceutical formulation suitable for sublingual administration in the treatment of obesity, Type II diabetes or a disorder related thereto.

According to embodiments of the present disclosure, the pharmaceutical formulation comprises:
5-10 mg of a glucagon-like peptide-1 receptor agonist peptide (GLP-1 RA-P) dissolved in 1.5-2.0 mL of a buffer solution;
   wherein,
the GLP-1 RA-P is selected from the group consisting of TE-8105, semaglutide, and tirzepatide, in which TE-8105 has the structure as depicted in FIG. 1;
the pharmaceutical formulation is suitable for sublingual administration and has a pH value of 5.5 to 7.5; and
the pharmaceutical formulation results in 2-10% bioavailability of the GLP-1 RA-P as compared to that achieved by subcutaneous injection upon being administered to a subject.

According to embodiments of the present disclosure, the pharmaceutical formulation is in the form of a liquid, a tablet, or a soft gel.

According to some embodiments of the present disclosure, the pharmaceutical formulation is in the form of a liquid and comprises 5-10 mg TE-8105 dissolved in 1.5-2.0 mL of a buffer solution.

According to other embodiments of the present disclosure, the pharmaceutical formulation is in the form of the liquid and comprises 5-6 mg semaglutide dissolved in 1.5-2.0 mL of a buffer solution.

According to further embodiments of the present disclosure, the pharmaceutical formulation is in the form of the liquid and comprises 5-10 mg tirzepatide dissolved in 1.5-2.0 mL of a buffer solution.

According to optional embodiments of the present disclosure, the pharmaceutical formulation further comprises 0.1% (w/v) peppermint oil.

According to optional embodiments of the present disclosure, the pharmaceutical formulation further comprises 5-10% gelatin and is in the form of soft gel.

According to optional embodiments of the present disclosure, the pharmaceutical formulation further comprises 1-10% crospovidone and is in the form of a tablet.

According to embodiments of the present disclosure, the disorder related to obesity or Type II diabetes is overweight, fatty liver disease, nonalcoholic steatohepatitis, diabetic cardiomyopathy, or atherosclerotic cardiovascular disease.

In another aspect, the present disclosure aims at providing a method for treating obesity, Type II diabetes or a disorder related thereto in a subject. The method includes:
sublingually administering an effective amount of a pharmaceutical formulation comprising a glucagon-like peptide-1 receptor agonist peptide (GLP-1 RA-P) to the subject, in which the pharmaceutical formulation is kept under the tongue of the subject for 5 to 10 minutes;
   wherein,
the GLP-1 RA-P is selected from the group consisting of TE-8105, semaglutide and tirzepatide, in which TE-8105 has the structure as depicted in FIG. 1;
the pharmaceutical formulation has a pH value of 5.5 to 7.5;
the pharmaceutical formulation is administered twice daily, once daily, once every two days, once weekly, or twice weekly; and
the sublingual administration of the pharmaceutical formulation achieves 2-10% bioavailability of the GLP-1 RA-P as compared to that achieved by subcutaneous injection.

According to embodiments of the present disclosure, the pharmaceutical formulation is in the form of a liquid, a tablet, or a soft gel.

According to certain embodiments of the present disclosure, the pharmaceutical formulation is in the form of a liquid and comprises 5-10 mg TE-8105 dissolved in 1.5-2.0 mL of a buffer solution and is administered once daily, twice daily, once every 2 days, once weekly, or twice weekly.

According to certain embodiments of the present disclosure, the pharmaceutical formulation is in the form of a liquid and comprises 5-6 mg semaglutide dissolved in 1.5-2.0 mL of a buffer solution and is administered once daily or once every 2 days.

According to certain embodiments of the present disclosure, the pharmaceutical formulation is in the form of a liquid and comprises 5-10 mg tirzepatide dissolved in 1.5-2.0 mL of a buffer solution, and is administered twice daily.

According to optional embodiments of the present disclosure, the pharmaceutical formulation further comprises 0.01% (w/v) peppermint oil.

According to optional embodiments of the present disclosure, the pharmaceutical formulation further comprises 5-10% gelatin and is in the form of a soft gel.

According to optional embodiments of the present disclosure, the pharmaceutical formulation further comprises 1-10% crospovidone and is in the form of a tablet.

According to embodiments of the present disclosure, the disorder related to obesity or Type II diabetes is overweight, fatty liver disease, nonalcoholic steatohepatitis, diabetic cardiomyopathy, or atherosclerotic cardiovascular disease.

In all embodiments of the present disclosure, the subject is a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic drawing depicting the structure of TE-8105 of the present disclosure;

FIG. 2. Comparison of blood glucose level in db/db mice between one subcutaneous (SC) injection and twice daily sublingual (SL) administrations for 3 days with TE-8105. (A) Changes in blood glucose levels with time; (B) Blood glucose levels for the first 3 time points treated with different SL dosages as compared to one SC dosage; and (C) The area under the curve (AUC) for blood glucose levels over the entire period of 168 hrs; the data is analyzed by two-way ANOVA with *P≤0.05, **P≤0.01, ***P≤0.0010, ****P≤0.00001;

FIG. 3 are 3 line graphs depicting time course of blood glucose levels from 3 individual mice in each group of FIG. 2;

FIG. 4. The additive and continual effects of repeated dosing of different dosages of TE-8105 via sublingual delivery in db/db mice. (A) Effects of 6 sublingual 2QD administrations of TE-8105 at 100 nmol/kg, 150 nmol/kg, and 250 nmol/kg on lowering blood glucose versus a single subcutaneous (SC) administration of 30 nmol/kg; (B) Blood glucose level after two SL administrations, with the third glucose time point taken prior to the third administration; (C) The AUC for blood glucose levels over the entire period of 168 hrs;

FIG. 5. Comparison of blood glucose level in db/db mice between one subcutaneous injection and twice daily sublingual administrations for 3 days with semaglutide. (A) Changes in blood glucose levels with time; (B) Blood glucose levels for the first 3 time points treated with different SL dosages as compared to one SC dosage; and (C) The AUC for blood glucose levels over the entire period of 144 hrs;

FIG. 6. Comparison of blood glucose level in db/db mice between one subcutaneous injection and twice daily sublingual administrations for 3 days with tirzepatide. (A) Changes in blood glucose levels with time; (B) Blood glucose levels for the first 3 time points treated with different SL dosages as compared to one SC dosage; and (C) The AUC for blood glucose levels over the entire period of 192 hrs;

FIG. 7. Blood glucose level in db/db mice with sublingual or oral treatment of TE-8105. (A) Changes in blood glucose levels with time; and (B) The AUC for blood glucose levels over the entire period of 192 hrs;

FIG. 8. Body weight of db/db mice with sublingual or oral treatment of TE-8105. (A) Changes in body weight of db/db mice with time; and (B) The AUC for body weight over the entire period of 192 hrs;

FIG. 9. Food intake in db/db mice with sublingual or oral treatment of TE-8105. (A) Changes in the food intake with time; and (B) The AUC for the food intake over the entire period of 192 hrs;

FIG. 10. Water intake of db/db mice with sublingual or oral treatment of TE-8105. (A) Changes in water intake of db/db mice with time; and (B) The AUC for water intake over the entire period of 168 hrs;

FIG. 11. Blood glucose level in db/db mice with sublingual treatment of different concentrations of TE-8105 for 5 days. (A) Changes in blood glucose levels with time, and the AUC for blood glucose levels over the entire period of 216 hrs; and (B) Changes in blood glucose levels in 72 hrs, and the AUC for blood glucose levels over the period of 72 hrs;

FIG. 12. Body weight of db/db mice with sublingual treatment of different concentrations of TE-8105 for 5 days. (A) Changes in body weight with time, and the AUC for body weight over the entire period of 216 hrs; and (B) Changes in body weight in 72 hrs, and the AUC for body weight over the period of 72 hrs;

FIG. 13. Food intake in db/db mice with sublingual treatment of different concentrations of TE-8105 for 5 days. (A) Changes in food intake with time, and the AUC for food intake over the entire period of 216 hrs; and (B) Changes in food intake in 72 hrs, and the AUC for food intake over the period of 72 hrs;

FIG. 14. Water intake of db/db mice with sublingual treatment of different concentrations of TE-8105 for 5 days. (A) Changes in water intake with time, and the AUC for water intake over the entire period of 216 hrs; and (B) Changes in water intake in 72 hrs, and the AUC for water intake over the period of 72 hrs;

FIG. 15. Blood glucose level in db/db mice with once daily sublingual treatment of TE-8105 for 5 days with varied sublingual treatment time. (A) Changes in blood glucose levels with time, and the AUC for blood glucose levels over the entire period of 216 hrs; and (B) Changes in blood glucose levels in 48 hrs, and the AUC for blood glucose levels over the period of 48 hrs;

FIG. 16. Body weight of db/db mice with once daily sublingual treatment of TE-8105 for 5 days with varied sublingual treatment time. (A) Changes in body weight with time, and the AUC for body weight over the entire period of 216 hrs; and (B) Changes in body weight in 72 hrs, and the AUC for body weight over the period of 72 hrs;

FIG. 17. Food and water intake in db/db mice with once daily sublingual treatment of TE-8105 for 5 days with varied sublingual treatment time. (A) Changes in food intake with time, and the AUC for food intake over the entire period of 216 hrs; and (B) Changes in water intake with time, and the AUC for water intake over the period of 216 hrs;

FIG. 18. Blood glucose level of db/db mice with once daily sublingual treatment of TE-8105 for 5 days with varied formulation pH values. (A) Changes in blood glucose level with time, and the AUC for blood glucose level over the entire period of 192 hrs; and (B) Changes in blood glucose level in 120 hrs, and the AUC for blood glucose level over the period of 120 hrs;

FIG. 19. Body weight of db/db mice with once daily sublingual treatment of TE-8105 for 5 days with varied formulation pH values. (A) Changes in body weight with time, and the AUC for body weight over the entire period of 192 hrs; and (B) Changes in body weight in 120 hrs, and the AUC for body weight over the period of 120 hrs;

FIG. 20. Food intake of db/db mice with once daily sublingual treatment of TE-8105 for 5 days with varied formulation pH values. (A) Changes in food intake with time, and the AUC for food intake over the entire period of 192 hrs; and (B) Changes in food intake in 120 hrs, and the AUC for food intake over the period of 120 hrs;

FIG. 21. Water intake of db/db mice with once daily sublingual treatment of TE-8105 for 5 days with varied formulation pH values. (A) Changes in water intake with time, and the AUC for water intake over the entire period of 192 hrs; and (B) 2.5 Changes in water intake in 120 hrs, and the AUC for water intake over the period of 120 hrs;

FIG. 22. Blood glucose level of db/db mice with twice daily sublingual treatment of TE-8105 or semaglutide for 3 days. (A) Changes in blood glucose level with time, and (B) the AUC for blood glucose level over the entire period of 128 hrs;

FIG. 23. Blood glucose level of db/db mice with twice daily sublingual treatment of TE-8105 or semaglutide at different doses for 3 days. (A) Changes in blood glucose level with time, and (B) the AUC for blood glucose level over the entire period of 128 hrs;

FIG. 24. Blood glucose level of db/db mice with once per day or once per two days sublingual treatment of TE-8105 or semaglutide for 7 days. (A) Changes in blood glucose level with time, and (B) the AUC for blood glucose level over the entire period of 271 hrs;

FIG. 25. Body weight of db/db mice with once per day or once per two days sublingual treatment of TE-8105 or semaglutide for 7 days. (A) Changes in body weight with time, and (B) the AUC for body weight over the entire period of 264 hrs;

FIG. 26. Food and water intake of db/db mice with once per day or once per two days sublingual treatment of TE-8105 or semaglutide for 7 days. (A) Changes in food intake over the entire period of 264 hrs; and (B) Changes in water intake over the entire period of 264 hrs;

FIG. 27. Blood glucose level of ZFDM rats with twice daily sublingual treatment of TE-8105 for 10 days. (A) Changes in blood glucose level with time, and (B) the AUC for blood glucose level over the entire period of 18 days;

FIG. 28. Body weight of ZFDM rats with twice daily sublingual treatment of TE-8105 for 10 days. (A) Changes in body weight with time, and (B) the AUC for body weight over the entire period of 18 days;

FIG. 29. Food intake of ZFDM rats with twice daily sublingual treatment of TE-8105 for 10 days. (A) Changes in food intake with time, and (B) the AUC for food intake over the entire period of 18 days;

FIG. 30. Water intake of ZFDM rats with twice daily sublingual treatment of TE-8105 for 10 days. (A) Changes in water intake with time, and (B) the AUC for water intake over the entire period of 18 days;

FIG. 31. Blood glucose level of db/db mice with twice daily sublingual treatment of TE-8105 liquid formulation containing peppermint oil and glycerol for 2 days. (A) Changes in blood glucose level with time, and (B) the AUC for blood glucose level over the entire period of 120 hrs;

FIG. 32. Blood glucose level of db/db mice with twice daily sublingual treatment of TE-8105 soft gel formulation for 3 days. (A) Changes in blood glucose level with time, and (B) the AUC for blood glucose level over the entire period of 168 hrs;

FIG. 33. Body weight of db/db mice with twice daily sublingual treatment of TE-8105 soft gel formulation for 3 days. (A) Changes in body weight with time, and (B) the AUC for body weight over the entire period of 168 hrs;

FIG. 34. Food and water intake of db/db mice with twice daily sublingual treatment of TE-8105 soft gel formulation for 3 days. (A) Changes in food intake with time, and the AUC for food intake over the entire period of 168 hrs; and (B) Changes in water intake with time, and the AUC for water intake over the entire period of 168 hrs;

FIG. 35. Blood glucose level of db/db mice with twice daily sublingual treatment of semaglutide soft gel formulation for 3 days. (A) Changes in blood glucose level with time, and (B) the AUC for blood glucose level over the entire period of 168 hrs;

FIG. 36. Body weight of db/db mice with twice daily sublingual treatment of semaglutide soft gel formulation for 3 days. (A) Changes in body weight with time, and (B) the AUC for body weight over the entire period of 168 hrs;

FIG. 37. Food and water intake of db/db mice with twice daily sublingual treatment of semaglutide soft gel formulation for 3 days. (A) Changes in food intake with time, and the AUC for food intake over the entire period of 168 hrs; and (B) Changes in water intake with time, and the AUC for water intake over the entire period of 168 hrs;

FIG. 38. Blood glucose level of db/db mice with twice daily sublingual treatment of TE-8105 tablets for 3 days. (A) Changes in blood glucose level with time, and (B) the AUC for blood glucose level over the entire period of 168 hrs;

FIG. 39. Body weight of db/db mice with twice daily sublingual treatment of TE-8105 tablets for 3 days. (A) Changes in body weight with time, and (B) the AUC for body weight over the entire period of 168 hrs;

FIG. 40. Food and water intake of db/db mice with twice daily sublingual treatment of TE-8105 tablets for 3 days. (A) Changes in food intake over the entire period of 168 hrs; and (B) Changes in water intake over the entire period of 168 hrs;

FIG. 41. Blood glucose level of db/db mice with twice daily sublingual treatment of semaglutide tablets for 3 days. (A) Changes in blood glucose level with time, and (B) the AUC for blood glucose level over the entire period of 168 hrs;

FIG. 42. Body weight of db/db mice with twice daily sublingual treatment of semaglutide tablets for 3 days. (A) Changes in body weight with time, and (B) the AUC for body weight over the entire period of 168 hrs;

FIG. 43. Food and water intake of db/db mice with twice daily sublingual treatment of semaglutide tablets for 3 days. (A) Changes in food intake over the entire period of 168 hrs; and (B) Changes in water intake over the entire period of 168 hrs;

FIG. 44. Blood glucose level in db/db mice with twice daily sublingual treatment of TE-8105 peppermint tablets for 3 days. (A) Changes in blood glucose level with time, and (B) the AUC for blood glucose level over the entire period of 144 hrs;

FIG. 45. Body weight in db/db mice with twice daily sublingual treatment of TE-8105 peppermint tablets for 3 days. (A) Changes in body weight with time, and (B) the AUC for body weight over the entire period of 144 hrs;

FIG. 46. Food intake in db/db mice with twice daily sublingual treatment of TE-8105 peppermint tablets for 3 days. (A) Changes in food intake with time, and (B) the AUC for food intake over the entire period of 144 hrs;

FIG. 47. Water intake in db/db mice with twice daily sublingual treatment of TE-8105 peppermint tablets for 3 days. (A) Changes in water intake with time, and (B) the AUC for water intake over the entire period of 144 hrs; and

FIG. 48 depicts the structure of azide-containing Aib-substituted GLP-1 agonist.

### DESCRIPTION

The present disclosure provides pharmaceutical formulations and uses thereof. The invention is based in part on the discovery that GLP-1 RA-Ps can be adsorbed by the sublingual mucosal epithelial layer.

For convenience, certain terms employed in the specification, examples and appended claims are collected here. Unless defined otherwise, all technical and scientific terms used herein have the same meaning as commonly understood by one of the ordinary skill persons in the art to which this invention belongs.

Ranges of values are disclosed herein. The ranges set out a lower limit value and an upper limit value. Unless otherwise stated, the ranges include all values to the magnitude of the smallest values (either lower limit value or upper limit value) and ranges between the values of the stated ranges.

The singular forms “a”, “and”, and “the” are used herein to include plural referents unless the context clearly dictates otherwise.

The term “effective amount” as used herein means an amount which provides a therapeutic or prophylactic benefit. According to embodiments of the present disclosure, administering one or more doses of the present formulation to a diabetic subject (e.g., a db/db mouse) will reduce the subject's blood glucose level to a normal low level.

The term “bioavailability of GLP-1 RA-P” as used herein refers to an estimated bioavailability of a sublingually administered GLP-1 RA-P in relative to that achieved by subcutaneous injection. Accordingly, the “bioavailability of GLP-1 RA-P” is expressed as a percentage of bioavailability achieves by subcutaneous injected GLP-1 RA-P. To estimate the bioavailability of sublingual delivery in relative to subcutaneous delivery in db/db mice, the blood glucose-lowering effects of two sublingual doses of varying amounts were compared with one subcutaneous dose of a specific amount at the 24-hour time point (just before the third sublingual dose). At this time, the subcutaneous dose would cause the blood glucose level to reach at a low (normal) level. The two sublingual doses of a specified amount would also cause the blood glucose level to reach at this low level. By comparing the combined effect of the two sublingual doses to the subcutaneous dose, the relative bioavailability is estimated. For example, if two sublingual doses of 200 nmol/kg achieved the same blood glucose-lowering effect as a subcutaneous dose of 50 nmol/kg at the 24-hour point, the bioavailability of sublingual administration is estimated to be approximately 50/400 (or 12.5%) in relative to subcutaneous administration. According to preferred embodiments of the present disclosure, a bioavailability range of 7-10% was observed for sublingual delivery of the present GLP-1 RA-P (i.e., TE-8105, semaglutide, or tirzepatide) in db/db diabetic, obese mice.

The term “subject,” “patient,” “individual” and the like are used interchangeably herein, and refer to a mammal, preferably human. The subject can be diagnosed with a condition. In some cases, the condition is obesity. In some cases, the condition is Type II diabetes. In some cases, the condition is a disorder related to obesity or Type II diabetes.

The subject can be a male or a female. The subject can be patients of any age. Often, the subject is a patient or other individual undergoing a treatment regimen, or being evaluated for a treatment regimen (e.g., treatment for the reduction of weight or hyperglycemia). However, in some instances, the subject is not undergoing a treatment regimen.

### 1. Pharmaceutical Formulation of GLP-1 RA-P for Sublingual Administration

Disclosed herein are injection-free pharmaceutical formulations for treating obesity, Type II diabetes or a disorder related thereto. The pharmaceutical formulation comprises GLP-1 receptor agonist peptides (GLP-1 RA-Ps) as its active ingredient and a pharmaceutically acceptable carrier, which may be a buffer, a polymer and the like, depending on the final form of the pharmaceutical formulation (e.g., liquid, tablets, gel, etc).

1.1 The present GLP-1 RA-P

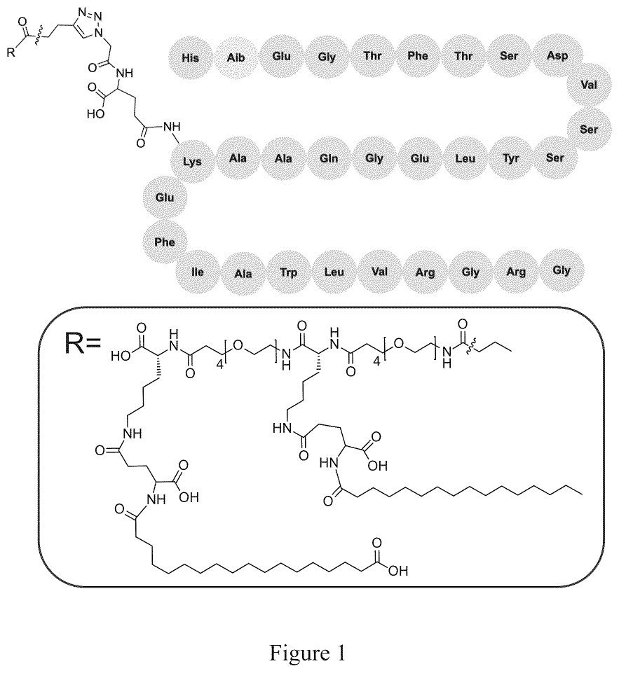

Native human GLP-1 is a peptide of about 30 amino acid residues and has very short half-life, approximately 2-5 min in human subjects. The GLP-1 RA-Ps, which have been successfully developed and approved for clinical use or are being developed typically have amino acid substitutions to minimize sensitivity to serum proteases, such as dipeptidyl peptidase-4 (DPP-4), so to increase serum half-life. According to embodiments of the present disclosure, examples of GLP-1 RA-Ps suitable for use in the present disclosure include, but are not limited to, TE-8105 (as depicted in FIG. 1), semaglutide, and tirzepatide. TE-8105 may be prepared in accordance with procedures described in Example 1 of the present disclosure or in Example 2 of US 2020/022498A1; semaglutide and tirzepatide may be obtained from commercial sources. All these GLP-1 RA-Ps share a common structural feature: they are each modified by conjugation with long-chain fatty acid(s). Both semaglutide and TE-8105 contain a peptide moiety consisting of 31 amino acid residues, which is a fragment (residues #7-37) derived from native human GLP-1, with artificially introduced amino acid substitutions to resist digestion by the serum protease, human dipeptidyl peptidase-4 (DPP-4). In both cases, the 20th amino acid residue is a lysine residue, whose ¿-amino group is conjugated with a long-chain C-18 diacid in semaglutide and a fatty acid bundle of C16 acid and C18 diacid in TE-8105. Tirzepatide, on the other hand, features a peptide moiety of 39 amino acid residues, designed to be homologous to both human GLP-1 and human glucose-dependent insulinotropic polypeptide (GIP). The 20th residue of tirzepatide is also a lysine residue, which is conjugated with a long-chain C20 diacid. The modification with long-chain fatty acids provides the GLP-1 RA-P with the following physiological benefits:
Enhanced association with serum albumin: The GLP-1 RA-Ps, once they cross the sublingual mucosal epithelial layer, reach the lamina propria and diffuse into the bloodstream. Their enhanced association with albumin in the interstitial space slows their diffusion into the bloodstream, while their binding to albumin in circulation extends their serum half-life, thereby contributing to increased bioavailability.
Improved affinity for mucosal epithelial cell membrane components, including lipids and proteins: This may facilitate the transport of these peptides across the epithelial cells.

### 1.2 Sublingual Formulations

The sublingual region in humans is relatively small, accommodating only a limited volume. Accordingly, the present disclosure aims at providing a sublingual formulation, which comprises an optimal amount of GLP-1 RA-P in maximum amount of a pharmaceutically acceptable carrier. Upon administration, it will produce therapeutic effect due to enhanced bioavailability of the GLP-1 RA-P conferred by the present formulation in relative to that achieved by subcutaneous injection.

### (i) Liquid Formulation

Several factors need to be considered when designing a liquid sublingual formulation that achieves maximum bioavailability of the drug molecules, which in turn produce desired therapeutic effects. The factors include, at least, the amount of drug molecules that may be absorbed in one sublingual dose, the maximum volume in one dose, and the maximum amount of drug carried in one dose (i.e., the solubility of the drug).

According to embodiments of the present disclosure, the maximum amount of GLP-1 RA-P that may be absorbed or transported across the oral mucosal membrane is about 3-5 mg/mL for TE-8105, and 3.2 mg/mL for semaglutide, which is affected by the pH of the buffer solution. According to further embodiments of the present disclosure, the volume suitable for one sublingual administration is found to be about 1.5 to 2.0 mL. In the case when the volume is greater than 2.0 mL, it would lead to overflow to the outside of the sublingual area in the oral cavity and the drug substance may enter gastric intestine tract due to swallow motions of the subject. Accordingly, the present liquid formulation is designed to comprise 5-10 mg of a GLP-1 RA-P dissolved in 1.5-2.0 mL of a buffer solution with a pH value between 5.5 to 7.5.

According to preferred embodiments of the present disclosure, the present pharmaceutical formulation is prepared by mixing a specified amount of GLP RA-P (i.e., TE-8105, semaglutide or tirzepatide) with a buffering agent (e.g., histidine) and an osmolality adjustor (e.g., mannitol) in deionized water until the GLP RA-P is completely dissolved, then adjusts the pH of the solution to a desired value, preferably between 5.5 to 7.5. For example, 5-10 mg of GLP-1 RA-P, such as 5, 6, 7, 8, 9 or 10 mg GLP-1 RA-P may be dissolved in 1.5-2.0 mL buffer solution, such as in 1.5, 1.6, 1.7, 1.8, 1.9 or 2.0 mL buffer solution, with a pH value being adjusted to about 5.5 and 7.5, such as 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In some instances, the GLP-1 RA-P is TE-8105, and the present formulation comprises 5-10 mg TE-8105 dissolved in 1.5-2.0 mL buffer solution. In other instances, the GLP-1 RA-P is semaglutide, and the present formulation comprises 5-6 mg semaglutide dissolved in 1.5-2.0 mL buffer solution. In further instances, the GLP-1 RA-P is tirzepatide, and the present formulation comprises 5-10 mg tirzepatide dissolved in 1.5-2.0 mL buffer solution.

Alternatively or optionally, in addition to the buffer described above, any buffer solution with osmolarity comparable to that of a human may also be used to prepare the present liquid formulation, such as phosphate buffered saline (PBS). Optionally or in addition, a preservative (e.g., phenol), a flavoring agent (e.g., peppermint oil), and/or an emollient (e.g., glycerol) may be added to the liquid formulation described above.

According to some embodiments of the present disclosure, the liquid formulation further comprises 0.1-0.5% (w/v) peppermint oil, such as 0.1, 0.2, 0.3, 0.4, or 0.5% (w/v) peppermint oil.

### (ii) Soft Gel Formulation

Optionally or in addition, the liquid formulation described above may be formulated with gelatin to produce soft gel, which is melted within 1-2 minutes upon delivery to the sublingual cavity.

According to embodiments of the present disclosure, the present liquid formulation is mixed with 5-10% (w/v) gelatin, such as 5, 6, 7, 8, 9, or 10% (w/v) gelatin to form a mixture, which will turn into a soft gel at low temperature (e.g., 4° C.), and can be easily picked up by a pair of forceps. Gelatin suitable for use in the present disclosure may derive from cold-water fish. In certain instances, the present liquid formulation is mixed with 5% (w/v) cold-water fish gelatin. In other instances, the present liquid formulation is mixed with 10% (w/v) cold-water fish gelatin.

(iii) Tablet Formulation

Alternatively or in addition, the liquid formulation described above may be formulated with a disintegrating polymer to produce a tablet, which upon contact with water or saliva will immediately disintegrate and release the drug molecule contained in the tablet.

According to embodiments of the present disclosure, the present liquid formulation is mixed with 1-10% (w/v) crospovidone (or polyvinyl polypyrrolidone), such as 1, 2, 3, 4, 5, 6, 7, 8, 9, or 10% (w/v) crospovidone to form a mixture, which will form a tablet after drying, and can be easily picked up by a pair of forceps and delivered to the sublingual region of a subject.

### 2. Methods of Treatment

The present formulations described in Section 1 of this paper are useful in the treatment of diseases or disorders related to hyperglycemia. Accordingly, another aspect of the present disclosure aims at providing a method of treating obesity, Type II diabetes or a disorder related thereto. The method comprises:
sublingually administering an effective amount of a formulation, which comprises a glucagon-like peptide-1 receptor agonist peptide (GLP-1 RA-P), to the subject, in which the formulation is kept under the tongue of the subject for 3 to 10 minutes;
   wherein,
the GLP-1 RA-P is selected from the group consisting of TE-8105, semaglutide and tirzepatide;
the formulation has a pH value of 5.5 to 7.5;
the formulation is administered once daily, twice daily, once every two days, once weekly, or twice weekly; and
the sublingual administration of the formulation achieves 2-10% bioavailability of the GLP-1 RA-P as compared to that achieved by subcutaneous injection.

According to embodiments of the present disclosure, the formulation may be in the form of a liquid, a tablet, or a soft gel, and is kept under the tongue of the subject for 3 to 10 minutes, such as3, 4, 5, 6, 7, 8, 9, or 10 minutes; preferably for 5 to 10 minutes, such as 5, 6, 7, 8, 9, or 10 minutes. In some cases, the formulation is kept under the tongue of the subject for 3 minutes. In some cases, the formulation is kept under the tongue of the subject for 5 minutes. In some cases, the formulation is kept under the tongue of the subject for 7 minutes. In some cases, the formulation is kept under the tongue of the subject for 10 minutes. In all embodiments of the present disclosure, blood glucose level, body weight, and food and water intake of the subject are reduced to a normal low level after administration of the formulation.

In some embodiments, the formulation is the liquid formulation described above and comprises 5-10 mg GLP-1 RA-P dissolved in 1.5-2.0 mL buffered solution. In certain instances, the liquid formulation comprises 5-10 mg TE-8105 dissolved in 1.5-2.0 mL buffered solution and is administered once daily, twice daily, once every 2 days, once weekly, or twice weekly. In certain instances, the liquid formulation comprises 5-6 mg semaglutide dissolved in 1.5-2.0 mL buffered solution and is administered twice daily or once per two days. In certain instances, the liquid formulation comprises 1-10 mg tirzepatide dissolved in 1.5-2.0 mL buffered solution and is administered twice daily.

In some embodiments, the formulation is the soft gel formulation described above and is prepared by mixing the present liquid formulation with 5-10% (w/v) gelatin. In some instance, the soft gel formulation is prepared by mixing the TE-8105 liquid formulation with 5% or 10% (w/v) gelatin and is administered at the same frequency as the formulation with the liquid. In some instances, the soft gel formulation is prepared by mixing the semaglutide liquid formulation with 5% or 10% (w/v) gelatin and is administered at the same frequency as the formulation with the liquid.

In some embodiments, the formulation is the tablet described above and is prepared by mixing the present liquid formulation with 1-10% (w/v) crospovidone. In some instances, the tablet is prepared by mixing the TE-8105 liquid formulation with 1% (w/v) crospovidone and is administered at the same frequency as the formulation with the liquid. In some instances, the tablet is prepared by mixing the semaglutide liquid formulation with 1% (w/v) crospovidone and is administered at the same frequency as the formulation with the liquid.

According to some embodiments of the present disclosure, as shown in the db/db diabetic, obese mouse model, sublingual doses of TE-8105 achieve bioavailability that is 7-8% of that achieved by subcutaneous injection. According to certain embodiments of the present disclosure, sublingual doses of semaglutide achieve bioavailability that is 7% of that achieved by subcutaneous injection. According to further embodiments of the present disclosure, sublingual doses of tirzepatide achieve bioavailability that is 10% of that achieved by subcutaneous injection. The bioavailability of 7-10% achieved by the present formulation and/or method represents an unexpected finding, as compared to two well-known peptide hormone drugs, oxytocin and desmopressin—both approved for sublingual delivery—each with a bioavailability of less than 1%.

According to embodiments of the present disclosure, the disorder related to obesity or Type II diabetes that may be treated by the present method is overweight, fatty liver disease, nonalcoholic steatohepatitis, diabetic cardiomyopathy, or atherosclerotic cardiovascular disease.

### EXAMPLES

### Materials and Methods

### Preparation of TE-8105, Semaglutide, and Tirzepatide Samples Four Use in the Present Study

The TE-8105 sample was prepared from the GMP-grade drug substance produced for Phase I and II human clinical trials by the CDMO company-WuXi STA Pharmaceutical Co. (Shanghai, China). The drug substance, initially in powder form, was formulated into a liquid preparation with a TE-8105 concentration of 5 mg/mL. The excipients used included histidine, phenol, mannitol, hydrochloric acid (HCl), and sodium chloride (NaCl), with a pH value of 7.0.

In certain experiments, the liquid formulation drug product at a concentration of 5 mg/mL, also manufactured at the CDMO, with the same excipients listed above, was utilized. Additionally, the commercial product Ozempic, intended for clinical use and containing 2 mg in a 1.5 mL vial (1.34 mg/mL), was purchased from a commercial source. The Ozempic formulation included the excipients dibasic sodium phosphate dihydrate, propylene glycol, phenol, hydrochloric acid, sodium hydroxide (for pH adjustment), and water for injection, with a final pH of 7.0.

Tirzepatide powder (Batch No. V423401), designated for research use only, was acquired from Invivo Chem (Libertyville, Illinois, USA). In our experiments, the tirzepatide powder was dissolved to a concentration of 5 mg/mL in a liquid formulation containing excipients sodium phosphate dibasic heptahydrate, sodium chloride, concentrated hydrochloric acid, sodium hydroxide (for pH adjustment), and water for injection. These excipients were used in the clinical product Mounjaro.

For each GLP-1 RA-P, dilutions were prepared to achieve different dosages using the respective liquid formulation solution as the diluting buffer.

The db/db Mouse Model for Studying Sublingual Delivery of GLP-1 RA-Ps and Bioavailability

The db/db mice are inbred mice carry a homozygous genetic mutation in the leptin receptor gene, leading to insatiable eating behavior and excessive weight gain, often reaching twice the normal body weight. Their blood glucose levels typically rise from below 200 mg/dL to above 400 mg/dL, and their HbA1c levels increase from below 6% to above 8%, or even higher. These parameters closely resemble those observed in human patients with type 2 diabetes and obesity. Further, blood glucose levels in db/db mice are highly sensitive to changes in GLP-1 RA-P drug concentrations, making glucose measurements a reliable indicator of the drug's availability in the bloodstream. In the experiments described in this study, the db/db mouse model was used to evaluate the efficiency of sublingual drug delivery, estimate bioavailability via sublingual versus subcutaneous administration, and examine the cumulative effects of repeated sublingual dosing.

For these experiments, 30 male db/db mice, aged 6 weeks and weighing 35-40 g, were used. After a two-week acclimation period in the laboratory, the mice were screened for weight and blood glucose levels. Mice with blood glucose levels between 300-400 mg/dL were selected for experiments. After receiving GLP-1 RA-P treatments, mice were rested for at least one week before subsequent testing. As the mice matured to 20-25 weeks of age, their body weight typically increased to 50-60 g.

Sublingual delivery of GLP-1 RA-P was performed as follows: The mouse was anesthetized using isoflurane vapor (2.5-3%, 700-800 mL/min) via a tube placed near its nose for 6-7 minutes. The mouse was positioned on its back on a soft Styrofoam board. To expose the sublingual area, the chin was gently pulled downward by looping a thin thread around the lower front tooth and securing it to the Styrofoam board with adhesive tape. A liquid formulation of GLP-1 RA-P (20-50 μL) was slowly applied to the sublingual area over a five-minute period. Once the anesthesia wore off, the mouse quickly regained activity, moving its tongue and mouth to swallow the administered liquid along with its saliva. During this interval, all or part of the GLP-1 RA-P was absorbed through the sublingual epithelial layer.

### The Zucker Fatty Diabetes Mellitus (ZFDM) Rat Model to Study Sublingual Delivery of GLP-1 RA-Ps and Bioavailability

The feasibility of administering TE-8105 via the sublingual route and its effects on glucose control and weight reduction were investigated in ZFDM rats. ZFDM rats are a subline with a missense mutation in the leptin receptor gene. These rats develop obesity within the first 6-12 weeks of life and diabetes between 10-20 weeks of age. They can grow to over 400 g in body weight and exhibit elevated non-fasting blood sugar levels of 400-500 mg/dL. This makes them a valuable model for studying potential treatments for obesity and diabetes.

ZFDM (fa/fa) rats aged 8-10 weeks, weighing about 150-200 g with non-fasting blood sugar of 120-150 mg/dL were used in the present study. The procedure for the sublingual delivery of GLP-1 receptor agonist peptides (GLP-1 RA-Ps) was conducted according to the procedures described above for db/db mice.

### Example 1: Synthesis of TE-8105

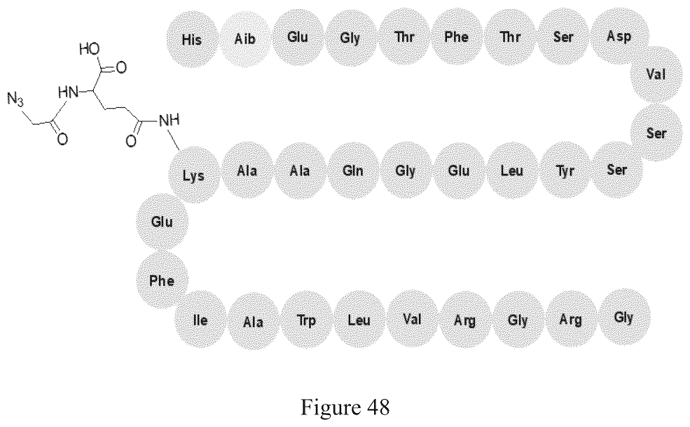

TE-8105 was synthesized by combining two structural parts, which are part I (azide-containing Aib-substituted GLP-1 agonist as depicted in FIG. 48) and part II (alkyne-2FA-C16-acid-C18-diacid as depicted below), by a copper (I) catalyzed alkene-azide click reaction.

### Part II: Alkyne-2FA-C16-Acid-C18-Diacid:

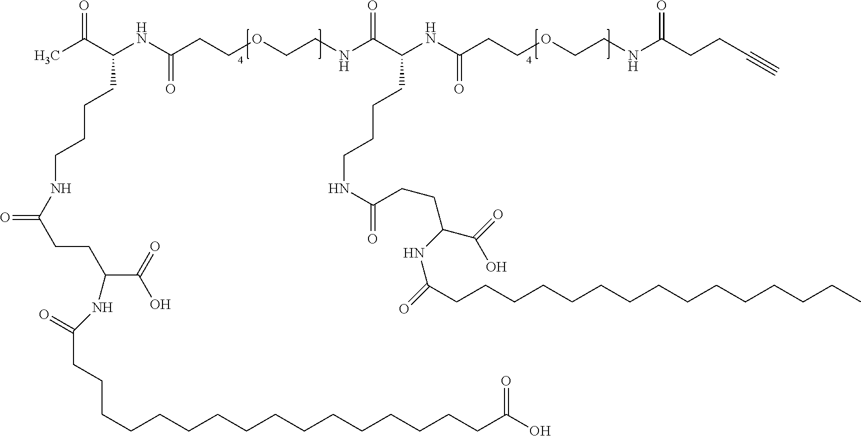

### 1.1 Synthesis of azide-Containing Aib-substituted GLP-1 agonist

Part I of TE-8105 was synthesized in accordance with procedures described in Chang et al (US 2020/022498A1). Briefly, the peptide part (SEQ ID NO: 1) was first synthesized by using solid-phase peptide synthesis (SPPS), then the alanine residue at position 2 of SEQ ID NO: 1 was replaced by 2-amino-isobutyric acid (Aib) residue to produce the Aib-substituted GLP-1 agonist (SEQ ID NO:2). Note that the Aib residue was included in the GLP-1 agonist to confer the molecule with resistance to dipeptidyl peptidase IV (DPP 4) degradation. Then, the γ-carboxy group of a glutamate residue was linked to the ε-amine group of lysine residue of the Aib-substituted GLP-1 agonist molecule (SEQ ID NO:2). Finally, the α-amino group of the glutamate residue was modified with an azidoacetyl group thereby producing the azide-containing Aib-substituted GLP-1 agonist molecule (or part I of TE-8105).

The peptide synthesis procedures involved a stepwise Fmoc SPPS using O-Benzotriazole-N,N,N′,N′-tetramethyl-uronium-hexafluoro-phosphate (HBTU)/N,N-diiso-propylethylamine (DIEA)/N,N-dimethylformamide (DMF) coupling chemistry, in which HBTU served as an in situ activating reagent for Fmoc protected amino acids and DIEA was used as an organic base during coupling. N^{α}-Fmoc, side-chain protected amino acids, and 2-chlorotrityl chloride resin (CTC resin) were used in the synthesis. The side-chain protection strategies were employed in the following Fmoc-protected amino acids: Fmoc-Arg (Pbf), Fmoc-Trp (Boc), Fmoc-Thr (OtBu), Fmoc-Lys (N-Dde), Fmoc-Tyr (OtBu), Fmoc-Glu (OtBu), Fmoc-Gln (Trt), Fmoc-Ser (OtBu), and Fmoc-His (Trt). Other Fmoc-protected amino acids were employed in the synthesis of GLP-1 agonist as following: Fmoc-Gly-OH, Fmoc-Val-OH, Fmoc-Leu-OH, Fmoc-Ala-OH, Fmoc-Ile-OH, Fmoc-Phe-OH, and Fmoc-Aib-OH. Abbreviations: Pbf, 2,2,4,6,7-Pentamethyldihydrobenzofuran-5-sulfonyl chloride; Boc, tert-butyloxycarbonyl; tBu, tert-butyl ether; Dde, 1-(4,4-dimethyl-2,6-dioxocyclohex-1-ylidene)ethyl; Trt, triphenylmethyl; TIS, triisopropylsilane; Fmoc, 9-fluorenylmethyloxycarbonyl.

For each coupling cycle, except the first, 3 mmol of Na-Fmoc-amino acid, 6 mmol of DIEA, and 2.85 mmol equivalents of HBTU were used. The Fmoc protecting group on the α-amine was removed using a 20% piperidine in dimethylformamide (DMF) solution (three times the volume of the peptide resin).

### Step (i): Initial Coupling

Peptide synthesis began by covalently linking the first amino acid to the resin. Fmoc-Gly-OH (1.0 mmol, 297.5 mg) and CTC resin (1.0 mmol, substitution=1.0 mmol/g, 1.0 g) were dissolved in dichloromethane (DCM), followed by the addition of DIEA (4.0 mmol). The mixture was agitated under nitrogen gas for 2 hours at 20° C. Methanol (MeOH, 1.0 mL) was then added as a capping reagent to react with unreacted carbocations on the CTC resin, and the mixture was stirred for 0.5 hours at 20° C.

### Step (ii): Washing

The methanol-containing capping solution was drained, and the resin was washed with DMF three times.

Step (iii): Fmoc Deprotection

The Fmoc protecting group on the CTC resin was removed by treating the resin with 20% piperidine in DMF for 0.5 hours at 20° C.

### Step (iv): Washing

After deprotection, the solution was drained, and the resin was washed with DMF five times.

### Step (v): Coupling the Second Amino Acid

The second amino acid, Fmoc-Arg (pbf))-OH (3 equivalents), and the activating agent HBTU were added to the resin. The mixture was agitated under nitrogen gas for approximately 1 hour. Steps (ii) to (v) were then repeated sequentially for each subsequent amino acid in the peptide sequence (SEQ ID NO:1). Each coupling reaction was monitored using the ninhydrin test.

### Cleavage and Purification

To cleave the side-chain-protected peptide from the CTC resin, a cleavage buffer (40.0 mL; 2.5% TIS/2.5% H₂O/2.5% 3-mercaptopropionic acid/92.5% trifluoroacetic acid (TFA)) was prepared and added to the flask containing the resin. The mixture was stirred at room temperature for 2 hours. The crude peptide was precipitated by adding it to cold isopropyl ether (500.0 mL) and centrifuged at 6000 rpm for 3 minutes. The crude peptide was washed with isopropyl ether two additional times and dried under vacuum for 2 hours.

The azido-containing Aib-substituted GLP-1 agonist (or part I of TE-8105) was purified by reverse phase HPLC on an Gemini C18 column (5 μm, 100 A) and Luna (10 μm, 100 A), using a mobile phase A of H₂O (0.075% trifluoroacetic acid in H₂O) and a mobile phase B of acetonitrile, a linear gradient of 17% to 47% acetonitrile over 60 minutes, at a flow rate of 20 mL/min and a column temperature of 25° C. A main peak at OD254 nm with a retention time of 11.65 minutes in the HPLC profile indicated the existence of the molecule of Aib-substituted and azido-containing GLP-1 agonist.

The Aib-substituted and azido-containing GLP-1 agonist was purified by reverse-phase HPLC using a Gemini C18 column (5 μm, 100 Å) and a Luna column (10 μm, 100 Å). The mobile phase consisted of A: H₂O with 0.075% trifluoroacetic acid and B: acetonitrile. A linear gradient from 17% to 47% acetonitrile was applied over 60 minutes at a flow rate of 20 mL/min and a column temperature of 25° C. The HPLC profile showed the main peak of the Aib-substituted and azido-containing GLP-1 agonist at OD254 nm with a retention time of 11.65 minutes, confirming the identity of the molecule.

### 1.2 Synthesis of alkyne-2FA-C16-acid-C18-diacid

In this Example, the part II structural component of TE-8105, i.e., an alkyne-containing linker unit featuring one palmitoleic acid and one octadecanedioic acid (denoted as “alkyne-2FA-C16-acid-C18-diacid”) was prepared. The peptide central core, alkyne-ethyl-Xaa4-K(C16)-Xaa4-K(C18-acid)-OH, contains two lysine (K) residues and an alkynylpropionyl group at its N-terminus. The spacers between the lysine residues and between the alkynylpropionyl group and its adjacent lysine residue are PEGylated amino acids with four ethylene glycol repeats (denoted as “Xaa4”). Two fatty acid chains with different lengths and terminal groups (C16-acid and C18-diacid) were conjugated to the lysine residues via amide bond formation between the carboxylic acid group (—CO₂H) of the fatty acids and the amine group of the lysine residues. This new molecule, “alkyne-2FA-C16-acid-C18-diacid,” was synthesized in accordance with the following procedures:

Step (i): Coupling of the first amino acid

The synthesis began by covalently linking the first amino acid to the resin. Fmoc-Lys(Dde)-OH (2.0 mmol, 1 equivalent) and CTC resin (2.0 mmol, 1 equivalent) were dissolved in dichloromethane (DCM, 10.0 mL). DIEA (8.0 mmol, 4 equivalents) was added to the mixture, which was swelled for 2 hours at 20° C. under nitrogen gas bubbling.

### Step (ii): Washing

The peptide-resin mixture was drained and washed with DMF three times.

Step (iii): Deprotection of the Fmoc group

The Fmoc protecting group was removed by adding 20% piperidine in DMF for 30 minutes.

### Step (iv): Washing

The resin was drained and washed with DMF five times.

### Step (v): Coupling of Fmoc-Glu-OtBu

Fmoc-Glu-OtBu (4.0 mmol, 2 equivalents) was coupled by adding DIEA (8.0 mmol, 4 equivalents) and Hexafluorophosphate azabenzotriazole tetramethyl uronium (HATU) (3.8 mmol, 1.9 equivalents) onto the resin under nitrogen gas bubbling for 30 minutes at 20° C. The product was washed with DMF five times.

Step (vi): Repeating steps for subsequent couplings

Steps (ii) to (vi) were repeated for coupling 18-(tert-butoxy)-18-oxooctadecanoic acid (2 equivalents). After coupling, 3% hydrazine/DMF solution was added to the peptide-resin mixture and incubated for 15 minutes. The product was washed with DMF (20 mL) five times.

### Coupling of Additional Components

Steps (ii) to (vi) were repeated for each of the following components:
Fmoc-NH-PEG4-CO₂H (1.5 equivalents)
Fmoc-Lys(Dde)-OH (2.0 equivalents)
Fmoc-NH-PEG4-CO₂H (1.5 equivalents)
Pent-4-ynoic acid (2.0 equivalents)

After the coupling of pent-4-ynoic acid, 3% hydrazine/DMF was added and incubated for 20 minutes. The product was washed with DMF five times. Steps (ii) to (vi) were then repeated for the coupling of Fmoc-Glu-OtBu (2.0 equivalents) and palmitic acid (2.0 equivalents).

### Cleavage and Purification

The side-chain-protected peptide was cleaved from the CTC resin using a cleavage buffer (1% TFA/99% DCM), which was added to the peptide resin at room temperature for 5 minutes twice. The crude peptide was concentrated under pressure and lyophilized, yielding 1.27 g of a yellow solid powder. The product was analyzed by liquid chromatography-mass spectrometry (LCMS). The retention time of the synthesized alkyne-containing linker unit with two aliphatic chains was 2.769 minutes, confirming the expected molecule.

### 1.3 Synthesis of TE-8105 (or 2FA-GLP-1 receptor agonist)

In this Example, the synthesis of a GLP-1 receptor agonist, referred to as “2FA-GLP-1 receptor agonist” or TE-8105, is described. TE-8105 was produced by coupling the Aib-substituted and azido-containing GLP-1 agonist of Example 1.1 with the alkyne-containing linker unit comprising one palmitoleic acid and one octadecanedioic acid of Example 1.2 via copper-catalyzed azide-alkyne cycloaddition (CuAAC) occurred between the azide and alkyne functional groups.

Briefly, a mixture of the Aib-substituted and azido-containing GLP-1 agonist (1.22 equivalents, TFA) and the alkyne-containing linker unit (1 equivalent, TFA) was prepared in DMF (4 mL). The solution was degassed and purged with nitrogen gas three times. Cu(I) (1 equivalent) and DIEA (4.0 equivalents) were added to the reaction mixture, which was then stirred at 20° C. for 10 minutes under a nitrogen atmosphere. Completion of the reaction was confirmed by LC-MS, where the retention time of the 2FA-GLP-1 receptor agonist (TE-8105) was observed at 1.784 minutes in the LC-MS profile.

The product, 2FA-GLP-1 receptor agonist, was purified by reverse-phase HPLC using a Luna C18 column (10 μm, 100 Å) and a Gemini C18 column (5 μm, 100 Å) in series. The purification employed a mobile phase A of water containing 0.075% trifluoroacetic acid and a mobile phase B of acetonitrile. A linear gradient from 30% to 60% acetonitrile over 60 minutes was applied at a flow rate of 20 ml/min, with the column temperature maintained at 30° C. The HPLC profile of the purified product displayed a main peak at OD214/OD254 nm with a retention time of 12.587 minutes, confirming the formation of TE-8105. The purified product was lyophilized, yielding 18.3 mg of white powder (10.1% yield, 96.4% purity, TFA).

### Example 2: The Bioavailability and the Additive and Continual Effects of Repeated Dosing of TE-8105 Via Sublingual Delivery in db/db Mice in a Twice Per Day (2QD) Regimen for 3 Days

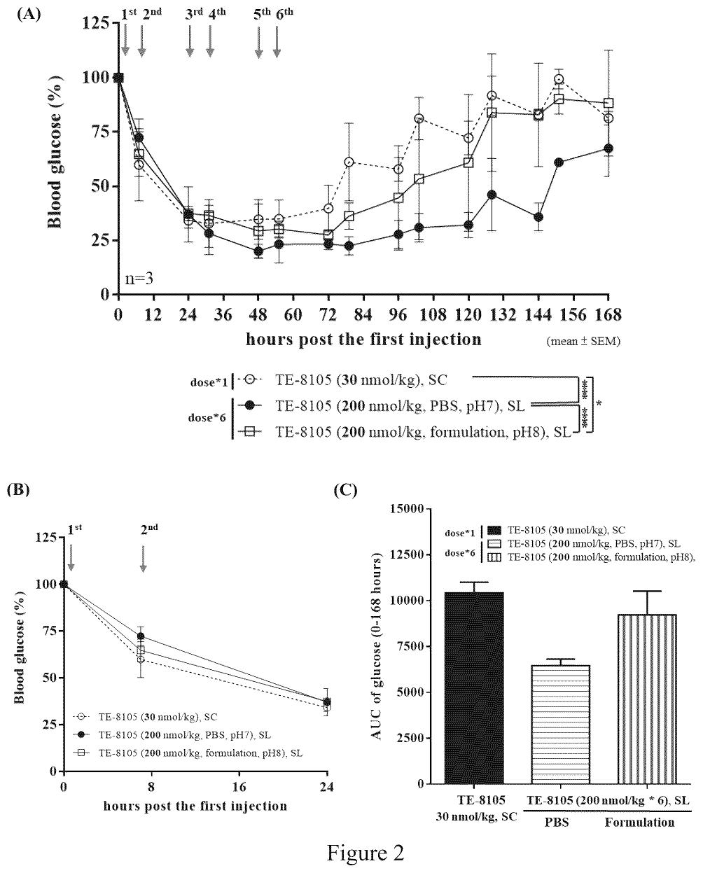

In this example, the bioavailability and the additive and continual effects of TE-8105 on blood glucose level was investigated using db/db mice model described in the “Materials and methods” section. To this purpose, two sublingual (SL) administrations of TE-8105 were made 7 hours apart in one day (or twice per day, 2QD) for consecutive three days. Blood samples were drawn prior to each TE-8105 administration and blood glucose level measured. Results are depicted in FIGS. 2, 3 and 4

It was found that blood glucose level could be lowered to normal levels and consecutive sublingual administrations of TE-8105 could maintain blood glucose at normal level; as the administration discontinued, blood glucose rose to abnormal levels in 2-3 days (FIG. 2A). Further, the glucose levels for the first three time points with different dosages of sublingual deliveries were compared with glucose levels achieved by a subcutaneous TE-8105 (2 doses of 200 nmol/Kg, 400 nmol/Kg total) (FIG. 2B). A total sublingual dose of 400 nmol/Kg produced a similar glucose-lowering effect after 24 hrs (the 3^{rd} time point, at 24 hours) as a 30 nmol/Kg subcutaneous dose. Thus, the bioavailability of sublingually administered TE-8105 was approximately about 7-8% (30/400) of that achieved by subcutaneously administration of TE-8105.

It was found that the area under the curve (AUC) for blood glucose over the entire period of 168 hours, comparing the effects of six sublingual doses versus one subcutaneous dose in decreasing blood glucose, indicated that consecutive 100 nmol/kg and 200 nmol/kg sublingual administration could achieve greater reduction of blood glucose than that of a subcutaneous administration (FIG. 2C).

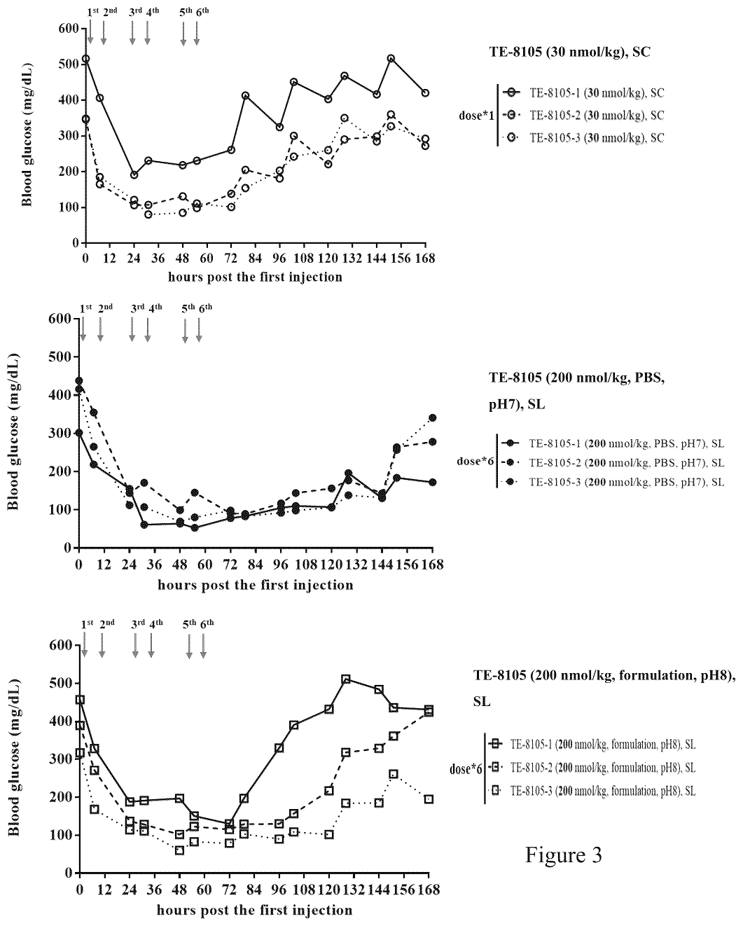

While the statistical analyses revealed some variability in blood glucose measurements at individual time points, the variability was attributed to inherent differences among individual mice or the bioassays per se. This conclusion is supported by the smooth, consistent patterns of blood glucose changes observed among the individual sets of three mice in each group (FIG. 3).

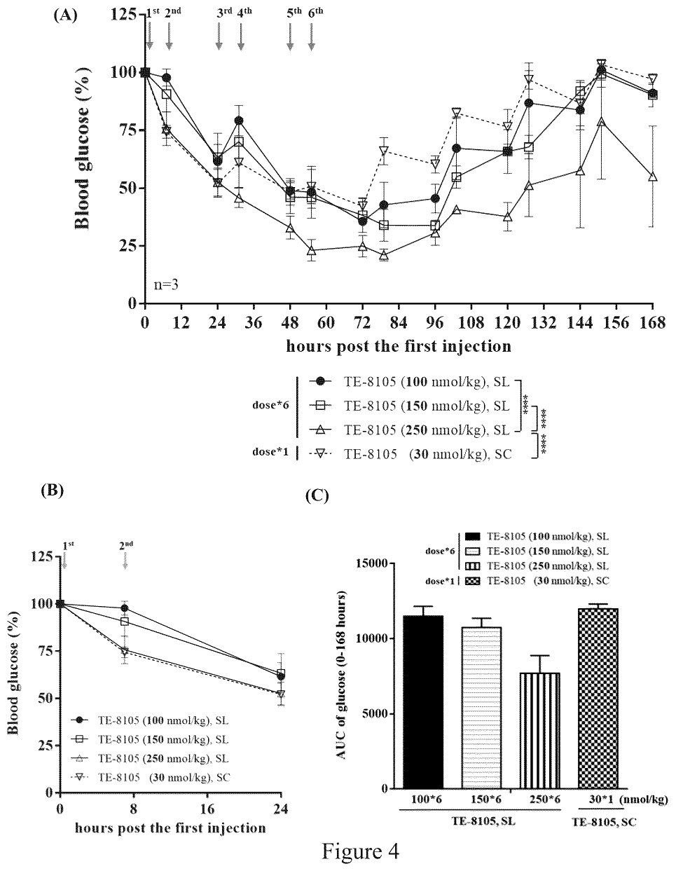

The additive and continual effects of repeated dosing of different dosages of TE-8105 via sublingual delivery in db/db mice are depicted in FIG. 4. In FIG. 4A, the effects of 6 sublingual 2QD administrations of TE-8105 at 100 nmol/kg, 150 nmol/kg, and 250 nmol/kg on lowering blood glucose versus a single subcutaneous (SC) administration of 30 nmol/kg were compared. FIG. 4B illustrates the effects after two sublingual administrations, with the third glucose time point taken prior to the third administration. FIG. 4C illustrates the dose-dependent effects on AUCs.

### Example 3. The Bioavailability and the Additive and Continual Effects of Repeated Dosing of Semaglutide Via Sublingual Delivery in db/db Mice in 2QD Regimen for 3 Days

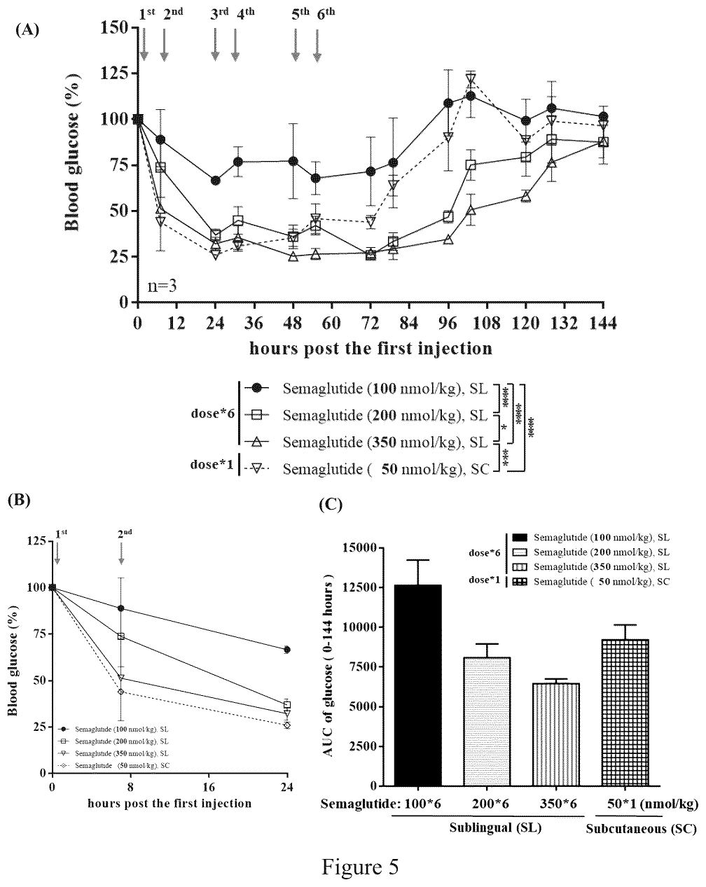

In this example, the bioavailability and the additive and continual effects of Semaglutide on blood glucose level was investigated using db/db mice model in accordance with similar procedures described in Example 2. Semaglutide was administered twice daily, 7 hours apart, for three consecutive days. Blood samples were collected prior to each administration to measure blood glucose levels. Results are depicted in FIG. 5.

It was found that consecutive sublingual administrations of semaglutide could effectively reduce and maintain blood glucose to normal levels. However, upon discontinuation, blood glucose levels quickly returned to abnormal levels within 1-2 days (FIG. 5A). Further, blood glucose levels at the first three time-points after different dosages of sublingual semaglutide were compared with levels achieved by a subcutaneous injection (FIG. 5B). It was found that with two doses of 350 nmol/kg (totaling 700 nmol/kg), the glucose-lowering effect at 24 hours (the 3rd time point) was nearly equivalent to that of a 50 nmol/kg subcutaneous injection. Thus, the bioavailability of sublingually administered semaglutide was approximately 7% (50/700) of the subcutaneous formulation. The dose-dependent effects of sublingual semaglutide on blood glucose reduction, as indicated by the AUC values are depicted in FIG. 5C.

### Example 4. The Bioavailability and the Additive and Continual Effects of Repeated Dosing of Tirzepatide Via Sublingual Delivery in db/db Mice in 2QD Regimen for 3 Days

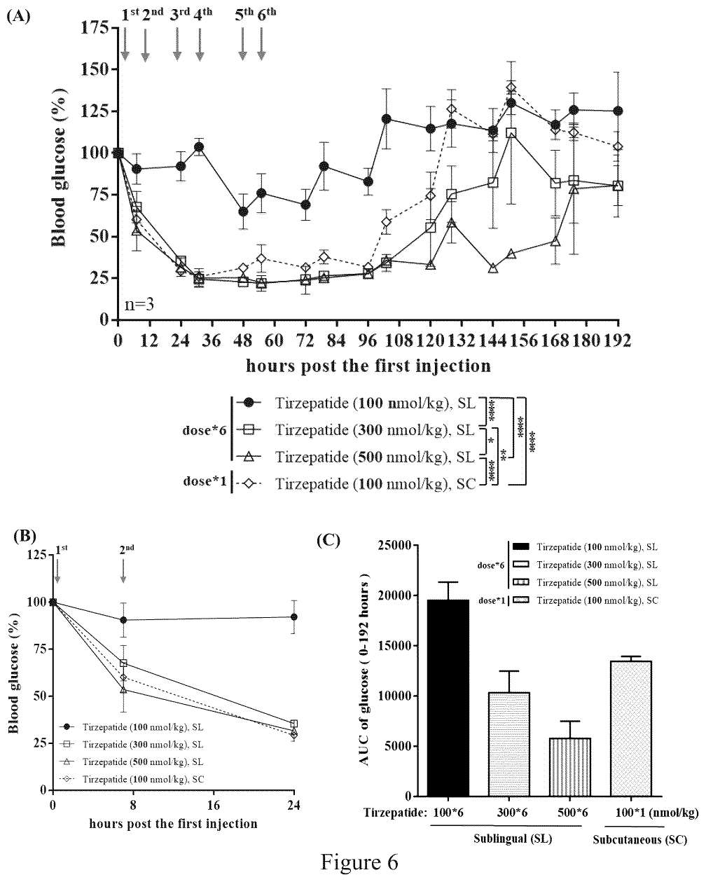

In this example, the bioavailability and the additive and continual effects of tirzepatide on blood glucose level was investigated using db/db mice model in accordance with similar procedures described in Example 2. To this purpose, two administrations of tirzepatide were made 7 hours apart in one day for consecutive three days. Blood samples were drawn prior to each tirzepatide administration and blood glucose measured. Results are illustrated in FIG. 6.

As depicted in FIG. 6A, blood glucose could be lowered to normal levels and that consecutive administrations of tirzepatide could maintain blood glucose at normal levels; however, as the administration discontinued, blood glucose rose to abnormal levels in 1-2 days. When the glucose levels for the first three time points after different dosages of sublingual deliveries were compared with glucose levels achieved by a subcutaneous tirzepatide, it was found that two sublingual doses of 500 nmol/kg (1,000 nmol/Kg total) resulted in a glucose reduction at 24 hrs (i.e., the 3rd time point), as compared to that of 100 nmol/Kg administered subcutaneously (FIG. 6B). Thus, the bioavailability of sublingually administered tirzepatide was about 10% (100/1000) of subcutaneously injected tirzepatide. The dose-dependent effects of sublingual tirzepatide on blood glucose reduction are provided as AUC values in FIG. 6C.

### Example 5. Comparison of Effects on Blood Glucose, Body Weight, and Food And Water Intake in db/db Mice Treated with Sublingual or Oral Administrations of TE-8105 Twice Daily for 3 Days

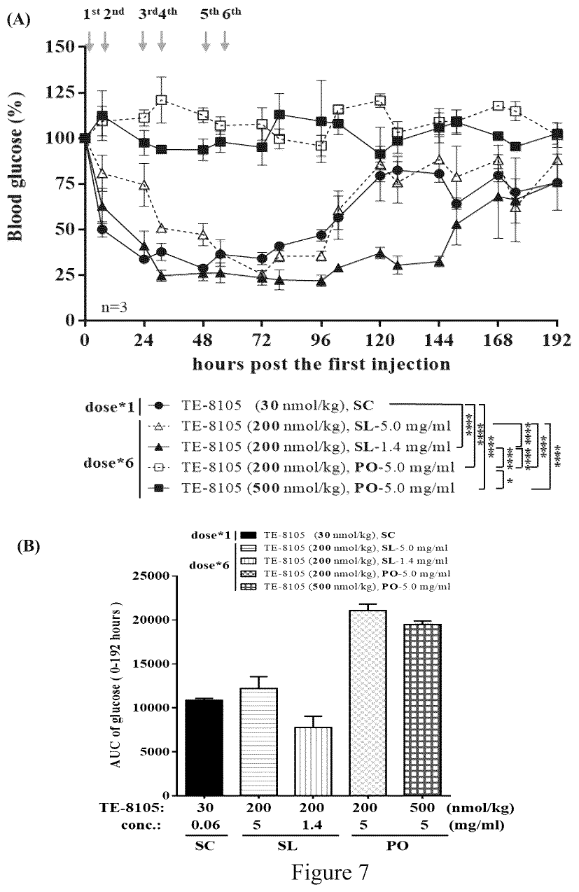

In this example, db/db mice treated with sublingual (SL) or oral (PO) administrations of TW-8105 twice daily for 3 days, and the blood glucose level, body weight and food and water intake of the mice were measured. For sublingual administration, the mice were anesthetized and the TE-8105 in liquid formulation was kept in the sublingual region for 5-10 minutes. For delivery through the mouth, the mouse was held in an upright position, and TE-8105 of the same amount but in two different concentrations in the liquid formulation was delivered to the mouth in 1-2 minutes with a pipet, and the mice would swiftly swallow the liquid. Results are provided in FIGS. 7 to 10.

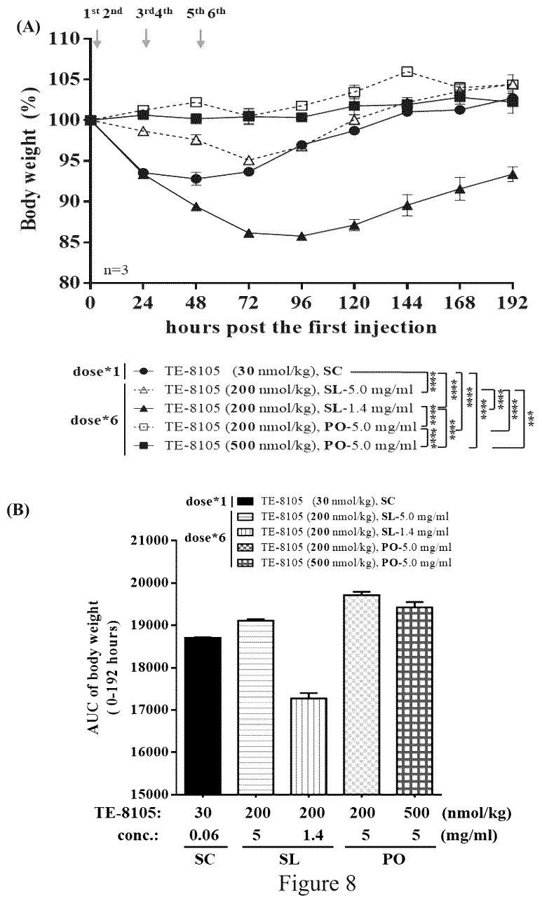

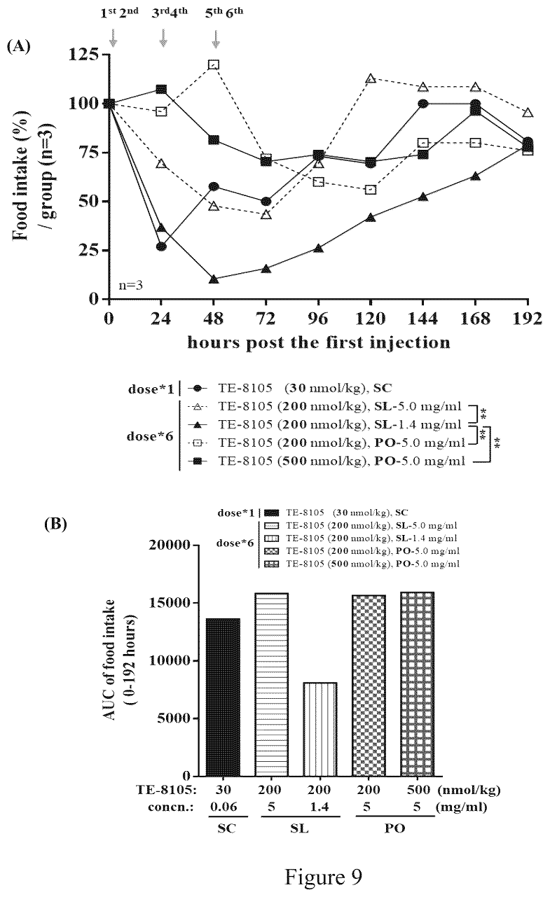

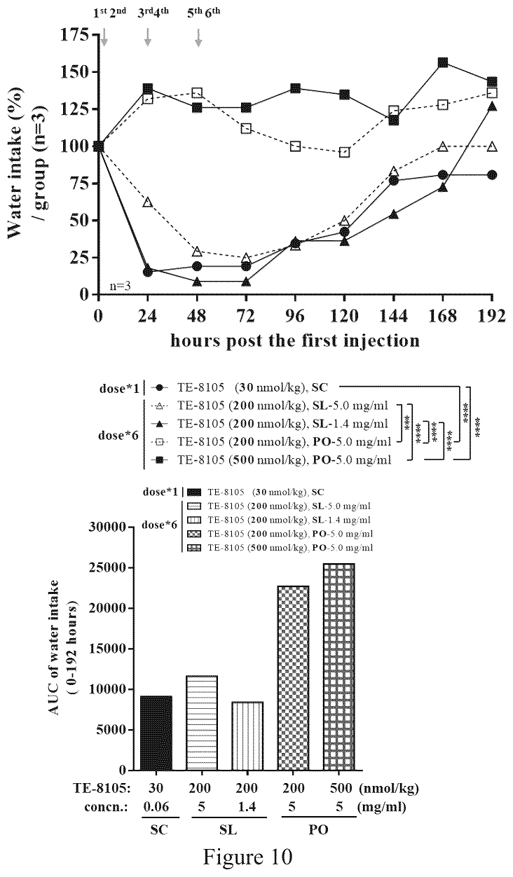

Results in FIG. 7 demonstrate that TE-8105 given through the mouth had no effects on reducing blood glucose level in the db/db mice, while TE-8105 administered sublingually could effectively lower blood glucose with the larger volume of the same amount being more effective. As to the effects on body weight, TE-8105 given through the mouth did not reduce body weight of the db/db mice; however, TE-8105, in particular, at 200 nmol/kg and 1.4 mg/ml given sublingually reduced body weight of the mice significantly (FIG. 8). Further, the reduction pattern for TE-8105 administered through different routes (SL or PO) on food intake in the db/db mice were very similar to those observed for body weight (FIG. 9). As to the effect on water intake, TE-8105 given through the mouth did not affect water intake, while TE-8105 administered sublingually or subcutaneously reduced water intake (FIG. 10).

### Example 6. Comparison of Effects on Blood Glucose, Body Weight, and Food and Water Intake in db/db Mice Treated with Varied Sublingual Concentrations of TE-8105 Once Daily for 5 Days

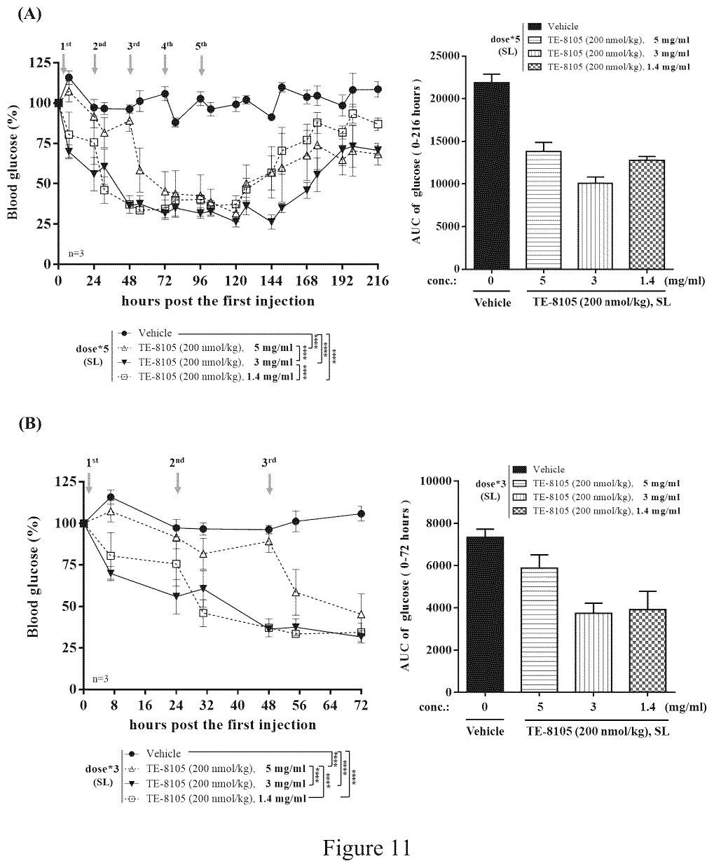

In this example, the concentration and volume of the liquid formulation of TE-8105 were examined. The sublingual region of an animal and a human is relatively small. To maximize sublingual transmucosal transport of a drug, it is sensible to utilize most of the sublingual mucosa. Thus, the volume of the liquid formulation is a relevant aspect. Moreover, the transmucosal transport presumably involves the interaction of the drug molecules in the liquid formulation with surface components of the mucosal epithelial cells. Therefore, there presumably exists an optimal concentration of the drug molecules. In this Example, TE-8105 of the same dosage, 200 nmol/kg at three concentrations 1.4, 3, and 5 mg/ml were prepared, and their absorption and subsequent pharmacodynamic effects were compared. Results are provided in FIGS. 11 to 14.

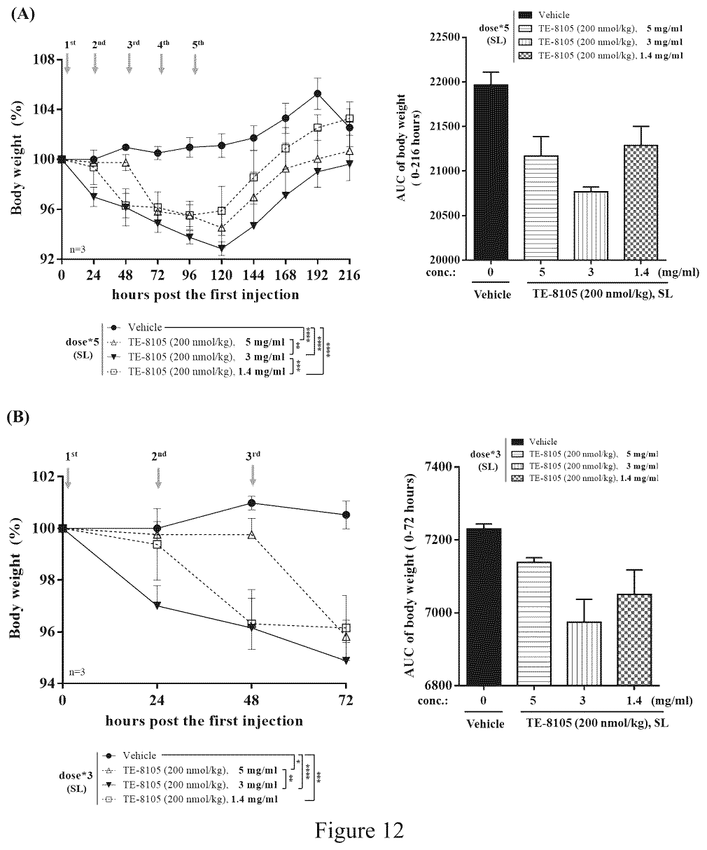

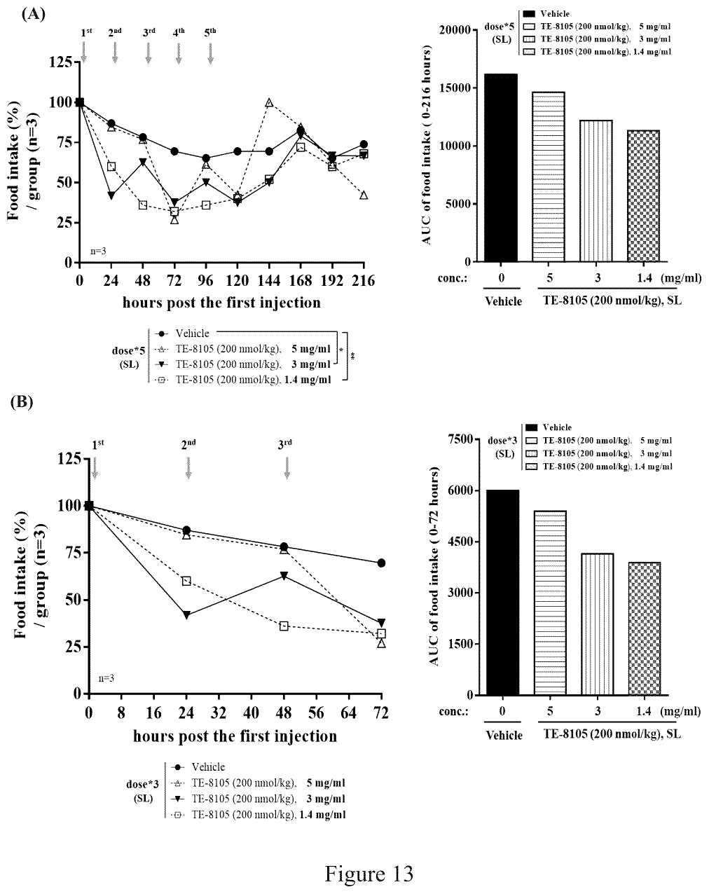

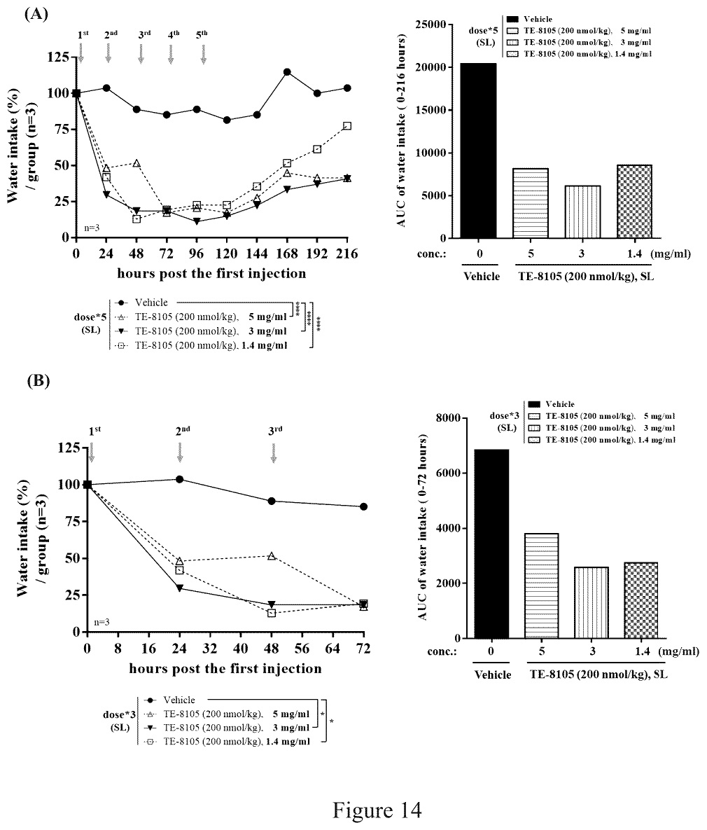

It was found that TE-8105 liquid formulation at three concentrations were all effective in lowering blood glucose, with the dose of 3 mg/ml being the most effective (FIG. 11). The kinetic patterns for the first 3 days are depicted in FIG. 9(B), the patterns were mostly the same as for longer periods. For the effect on the reduction of body weight, TE-8105 liquid formulation at three concentrations all exhibited the effect on reducing body weight of the obese db/db mice, with the dose of 3 mg/ml being the most effective (FIG. 12). Further, TE-8105 at three concentrations all reduced food intake and water intake in db/db mice, however, the effects among them were not statistically significant (FIGS. 13 and 14).

### Example 7. Comparison of the Effects on Blood Glucose, Body Weight, and Food and Water Intake in db/db Mice Among Different Lengths of Sublingual Administering Time of TE-8105 Given Once Daily for 5 Days

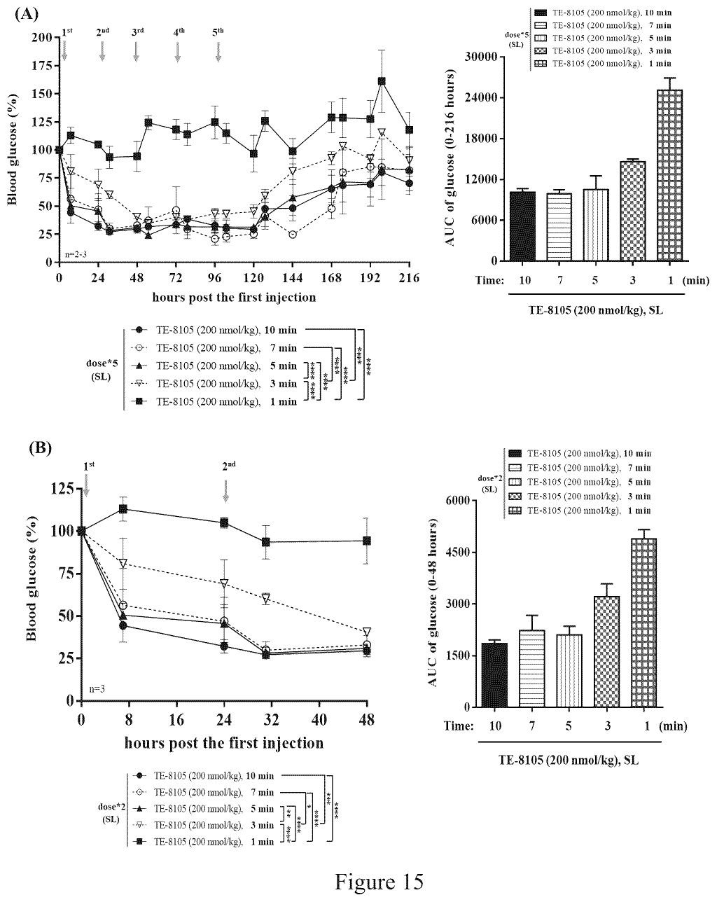

It is a general practice for a patient to hold the tongue and mouth still for 5 to 10 minutes when taking a medicine via sublingual administration, so that the medicine has time to contact the mucosal epithelial cells and get absorbed. Accordingly, the efficiency of TE-8105 crossing the sublingual epithelial layer of db/db mice was investigated in this example, in which the TE-8105 liquid formulation was held in place in the anesthetized mice for 1, 3, 5, 7, and 10 minutes, before the anesthesia was relieved. Results are provided in FIGS. 15 to 17.

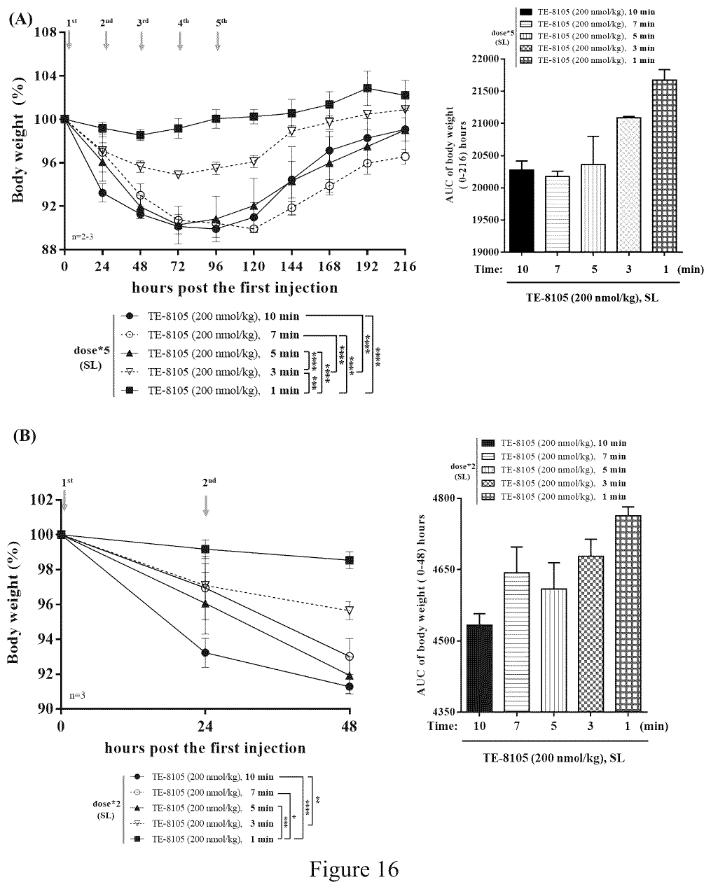

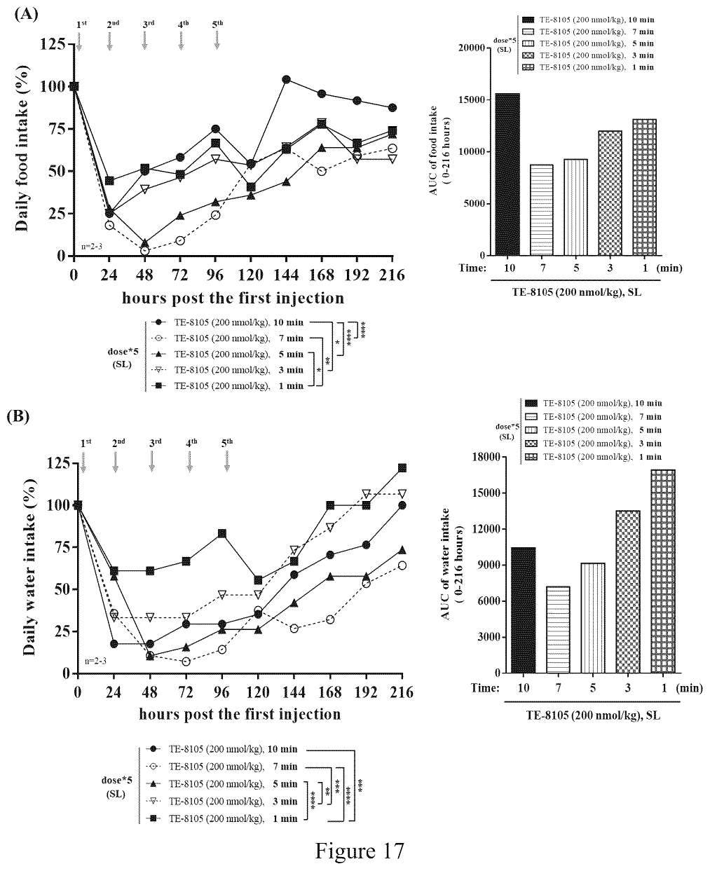

It was found that exposing the sublingual region to TE-8105 for 1 minute produced blood glucose reduction effect that was substantially below optimal, and 3 minutes was somewhat suboptimal for the TE-8105 to be absorbed, and the effects levelled off for exposure times being 5, 7, and 10 minutes (FIG. 15). Reduction of body weight was found in db/db mice treated with TE-8105 in the sublingual region over 5, 7, or 10 minutes, but not over 1 or 3 minutes (FIG. 16). Further, TE-8105 could cause food and water intake reduction when the TE-8105 liquid formulation was sublingually delivered to the db/db mice over 5 or 7 minutes (FIG. 17).

### Example 8. Comparison of the Effects on Blood Glucose, Body Weight, and Food and Water Intake in db/db Mice Among Sublingually Administered TE-8105 of Different pH Given Once Daily for 5 Days

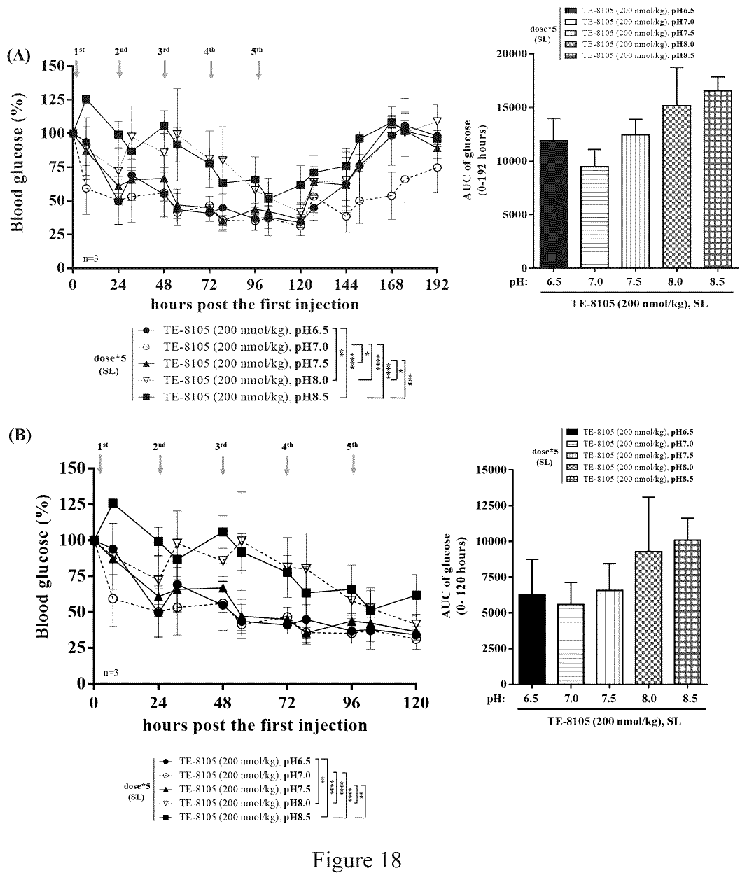

In this example, the effect of pH of the liquid formulation on the efficiency of sublingual transmucosal absorption of TE-8105 was investigated. To this purpose, TE-8105 liquid formulations with pH 6.5, 7.0, 7.5, 8.0, and 8.5 were prepared, and administered to db/db mice sublingually once daily for 5 days. Results are provided in FIGS. 18 to 21.

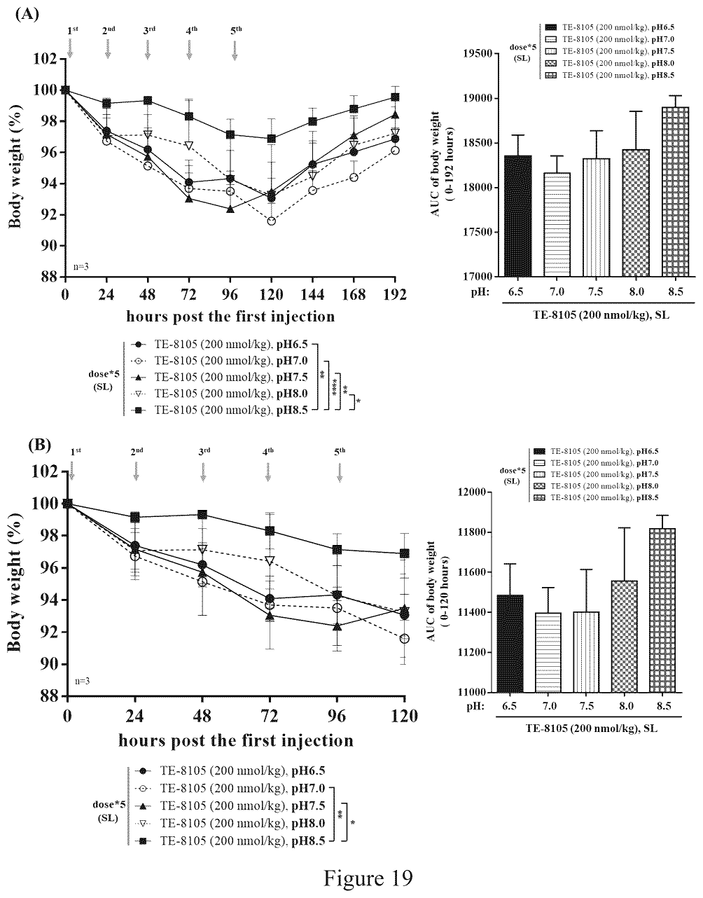

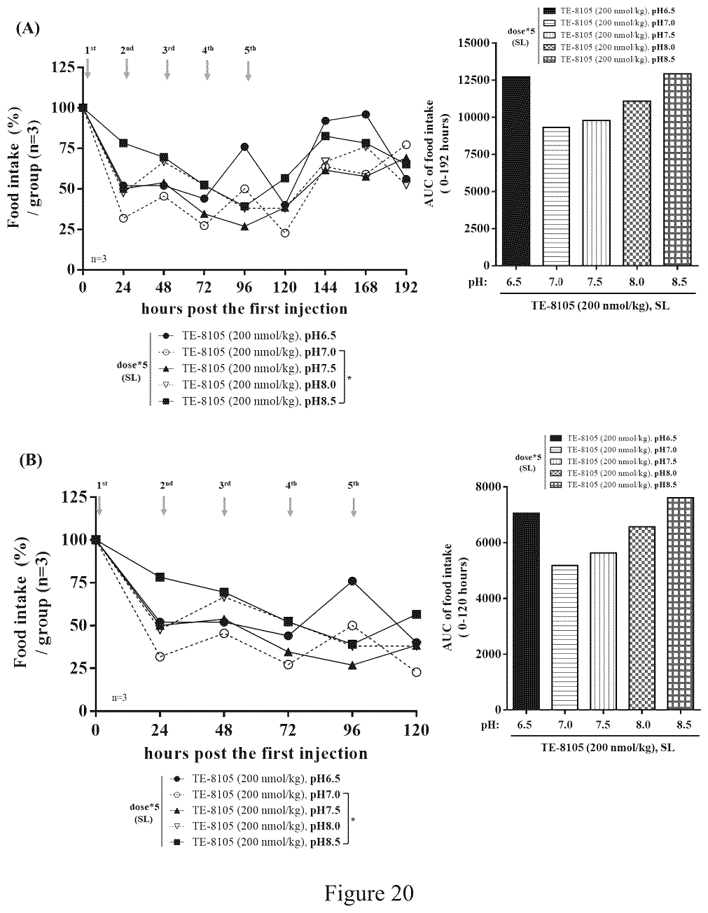

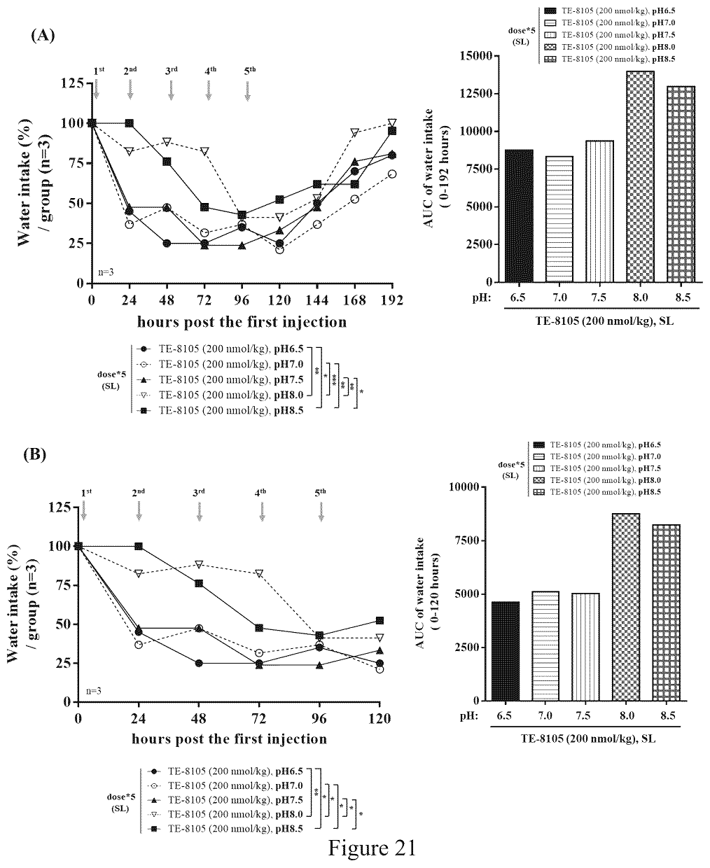

As illustrated in FIG. 18, TE-8105 liquid formulation with pH 7.0 gave the best results in terms of reducing blood glucose level, the formulation of pH 7.5 being the second effective, and those with pH 6.5, 8.0, and 8.5 being less than satisfactory. Further, TE-8105 liquid formulation at pH7.0 and 7.5 could produce the most effective body weight reduction, while those with pH6.5, 8.0, and 8.5 could not (FIG. 19). In addition, TE-8105 at pH 7.0 exhibited the best effect on reducing food intake (FIG. 20); while TE-8105 liquid formulations at pH 7.0 and pH 7.5 exhibited statistically significant differences from those at other pH values in terms of reducing water intake (FIG. 21).

### Example 9. The Cumulative and Trailing Off Effects on Blood Glucose Control Between TE-8105 and Semaglutide in db/db Mice with Sublingual Delivery in a 2QD Regimen

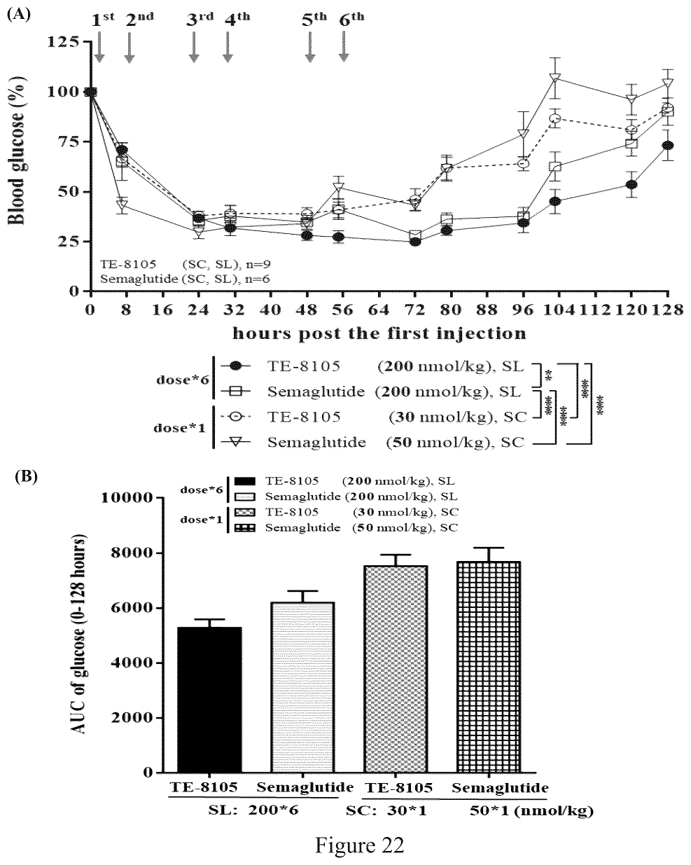

Both TE-8105 and semaglutide were found to have similar bioavailability of 7-8% via sublingual administration, as described in Examples 2 and 3. In the present example, whether the relative potency and half-lives between TE-8105 and semaglutide affect their effectiveness and kinetics on blood glucose control in db/db mice were investigated. To this purpose, db/db mice were treated with TE-8105 or semaglutide sublingually twice per day (2QD) at the dose of 200 nmol/Kg, and blood glucose levels over a period of 128 days were measured. Results are illustrated in FIG. 22.

It was evident that semaglutide could keep blood glucose at the normal levels as that of TE-8105 with frequent administration; however, the blood glucose returned to abnormal levels sooner with semaglutide than with TE-8105, during the trailing off period after the dosing discontinued (FIG. 22).

### Example 10. Effects of Different Dosages of TE-8105 or Semaglutide on Blood Glucose Level in db/db Mice in 2QD Regimen

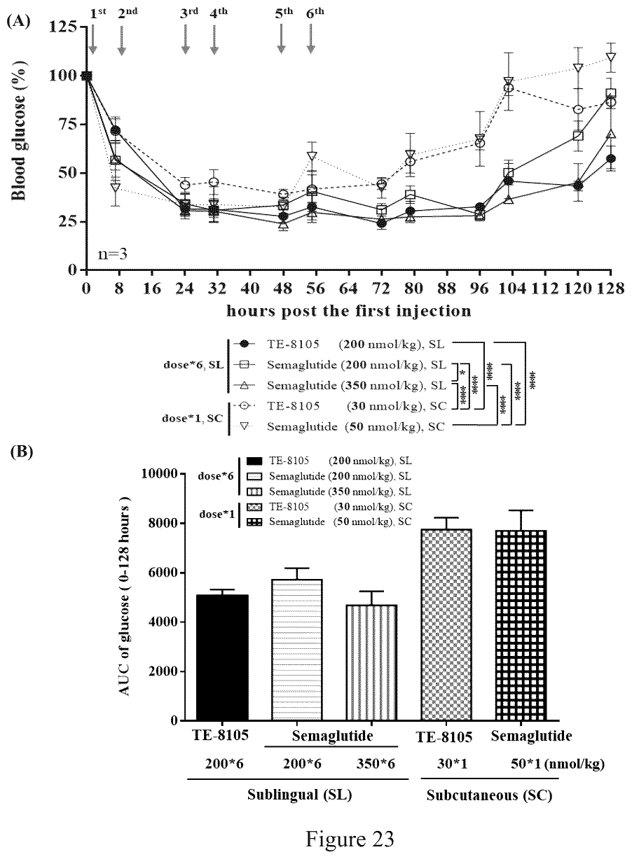

In this example, effects of different dosages of TE-8105 and semaglutide administered sublingually twice per day on blood glucose level in db/db mice were investigated. Results are illustrated in FIG. 23.

It was found that a higher dose of semaglutide (350 nmol/kg) than TE-8105 (200 nmol/kg) was needed to reduce blood glucose to the same levels in twice per day (2QD) sublingual administration regimen (FIG. 23). These observations were in parallel with the subcutaneous injection of the two GLP-1 RA-Ps, as 50 nmol/kg of semaglutide and 30 nmol/Kg of TE-8105 exhibited similar effects in lowering blood glucose.

### Example 11. Effects of Sublingual TE-8105 and Semaglutide on Blood Glucose Control, Body Weight Reduction, and Water and Food Intake in db/db Mice in Prolong Once Per Day (QD) or Once Per 2 Days (Q2D) Regimens

As described in Examples 2 and 3, one major purpose of this paper was to estimate the bioavailability of GLP-1 RA-Ps between sublingual delivery and subcutaneous delivery, hence the first 2 sublingual doses were administered only a few hours (7 hours) apart on the first day, so that the clearance of the drugs was not substantial. In this example, the feasibility and relative advantage of sublingual delivery between TE-8105 and semaglutide in once per day (QD) or once per 2 days (Q2D) regimens were investigated. The rationale is that semaglutide has been approved for marketing by regulatory agencies for treating type-2 diabetes and obesity. An injection-free, oral formulation of semaglutide (Rybelsus) has also been approved for marketing, setting a status-of-the art benchmark for the field of drug delivery for GLP-1 RA-Ps.

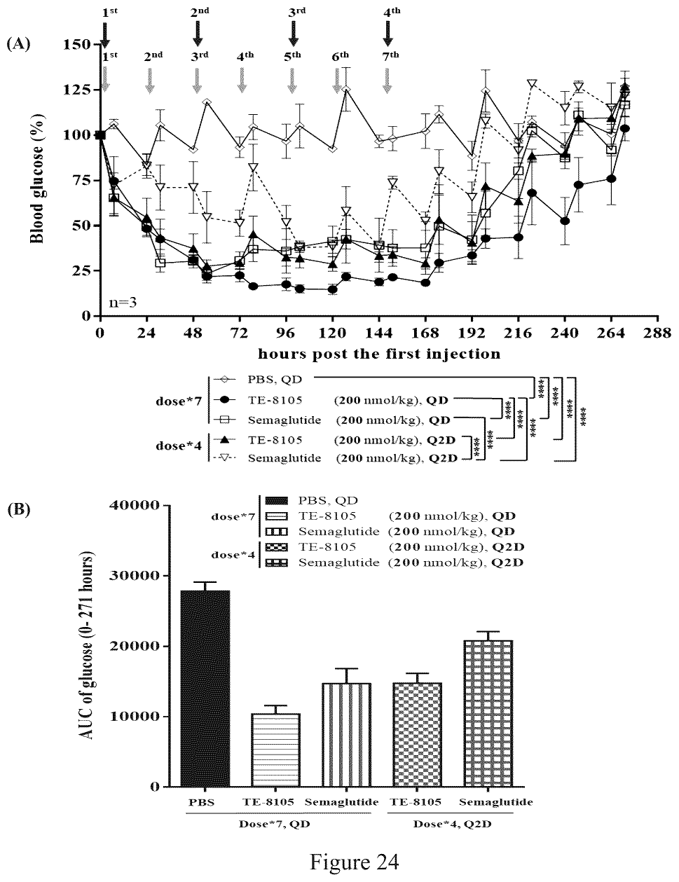

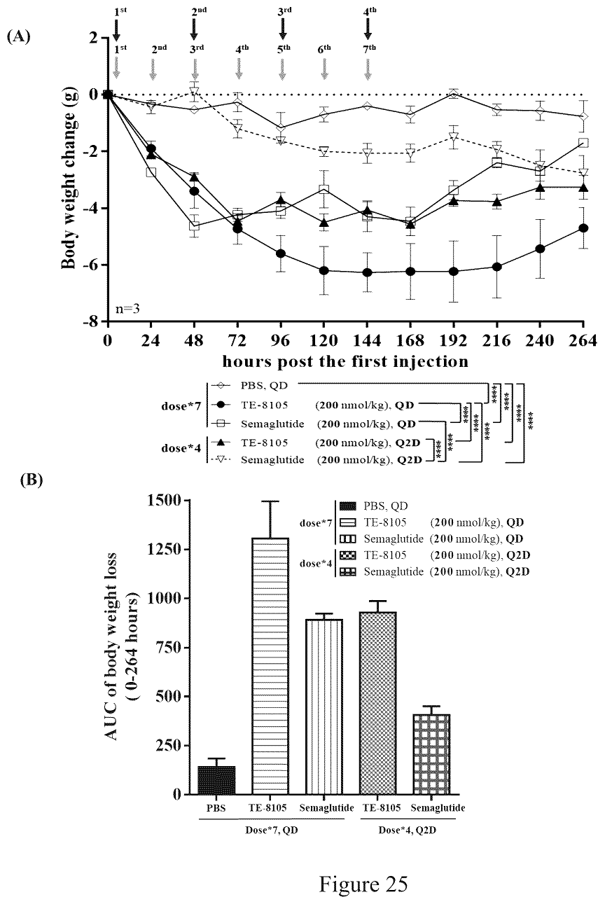

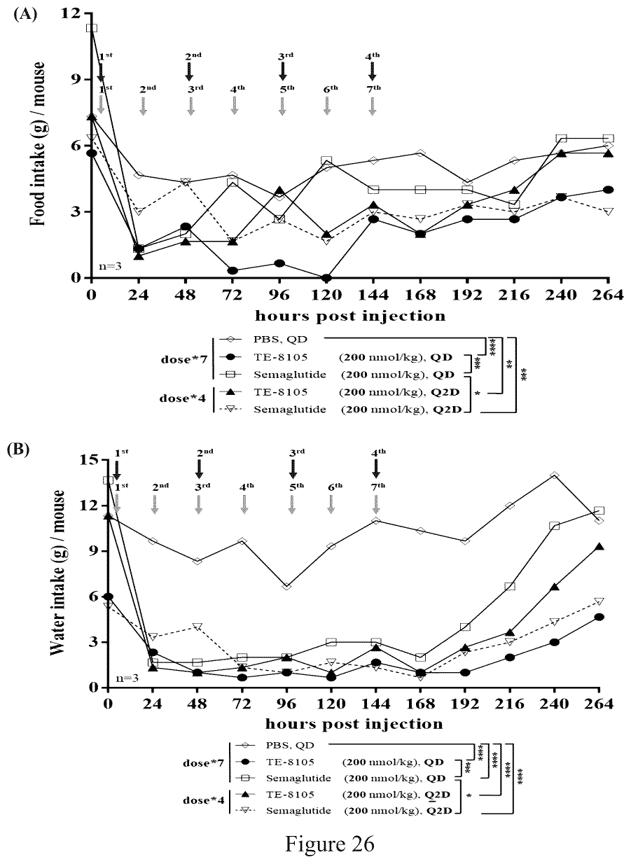

To simulate potential dosing schemes in human subjects, db/db mice were administered with TE-8105 or semaglutide once daily (QD) or once per 2 days (Q2D) over a week period, for a total of 7 or 4 administrations, respectively. Blood glucose levels were measured every day (FIG. 24A) and relative AUCs of blood glucose levels over the entire period were compared (FIG. 24B), body weight for the different groups (FIG. 25), and food intake and water intake (FIG. 26) were measured every day for 11 days, including the trailing off days after the last drug administration. The results show that while both semaglutide and TE-8105 at 200 nmol/Kg could control blood glucose, lower body weight, and reduce water and food intake over the extended once per day (QD) regimen; however, TE-8105, but not semaglutide, could still control blood glucose, lower body weight, and reduce water and food intake in the Q2D regimen. Based on the experience of subcutaneous treatment with GLP-1 RA-Ps, a smooth Q2D treatment profile can convert to smooth QW treatment profile in human subjects.

### Example 12. Effects of TE-8105 on Blood Glucose, Body Weight, and Food and Water Intake in ZFDM Rats in 2QD Regimen for 10 Days

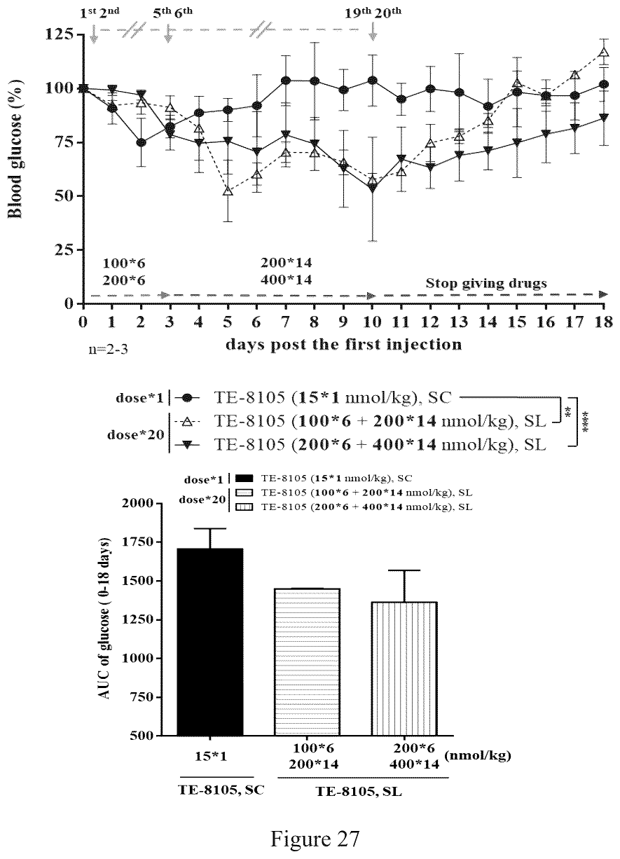

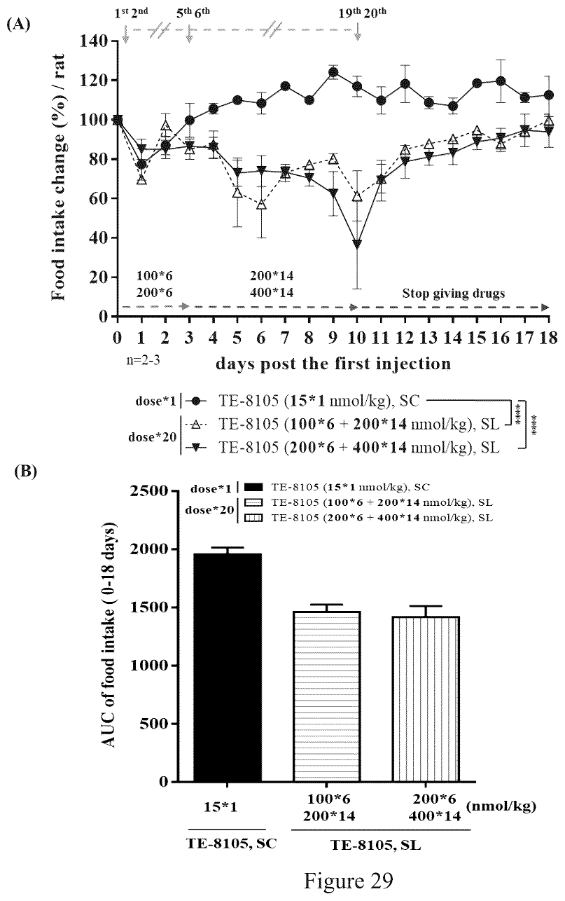

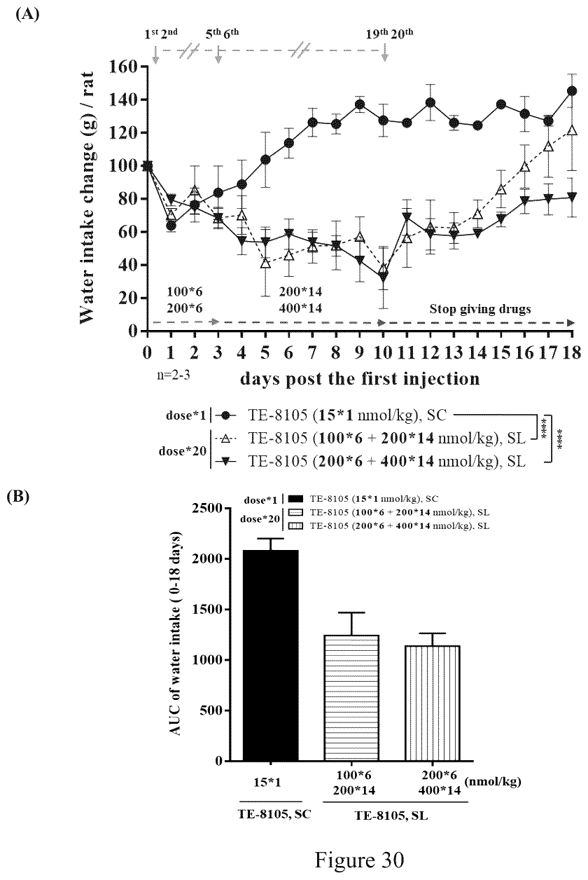

In this example, effects of TE-8105 on the blood glucose level, body weight, food and water intake in ZFDM rats were investigated. Results are provided in FIGS. 27 to 30.

It was found that a single subcutaneous injection of TE-8105 at 15 nmol/kg reduced the blood glucose by 25% in 2 days, before it returned gradually over a few days. Two regimens of repeated TE-8105 sublingual treatments were provided, in which (1) 200 nmol/kg for 6 doses and 200 nmol/kg for 14 doses, and (2) 200 nmol/kg for 6 doses and 400 nmol/kg for 14 doses. Treatments were administered twice daily for 10 days, after the end of the treatments, and the rats were continuously monitored for another 8 days.

It was found that with a subcutaneous injection of TE-8105, the body weight of rats dipped for one day and then kept increasing for next 16 days. On the other hand, the two sublingual TE-8105 regimens caused gradual decrease in body weight during the 10 days of TE-8105 treatments, and the body weight resumed increased for additional 8 days after discontinue of the treatment (FIG. 27).

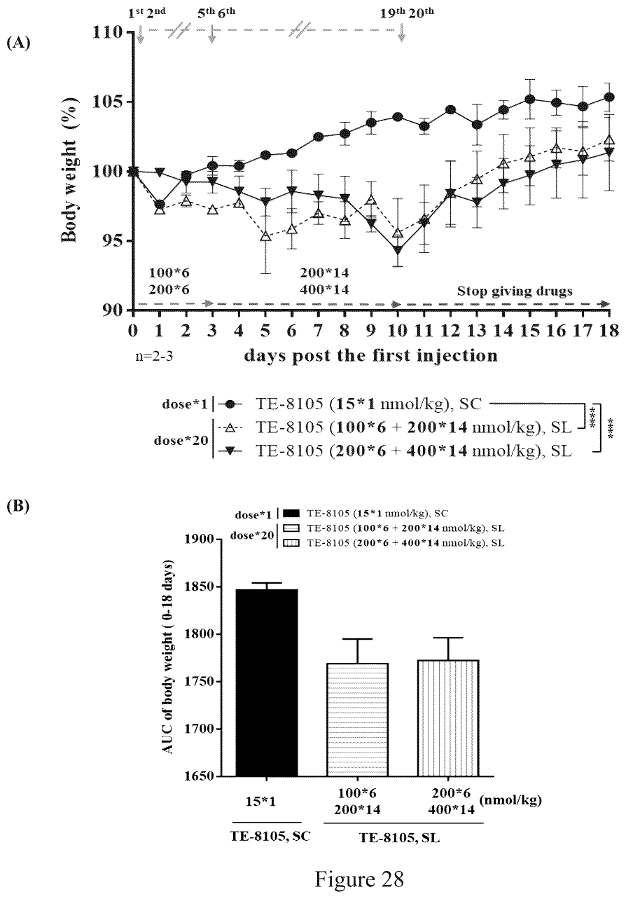

The patterns of changes in food and water intake upon receiving the subcutaneous injection and the two sublingual regimens almost mirrored those of body weight changes. The AUC plots further demonstrated that both the two sublingual regimens could reduce blood glucose, body weights, and food intake to greater extents than those achieved by one single subcutaneous injection (FIGS. 28 to 30).

### Example 13. Effects of Sublingual Administered TE-8105 Formulation Comprising Peppermint Oil and Glycerol on Blood Glucose Levels in db/db Mice in 2QD Regimen for 2 Days

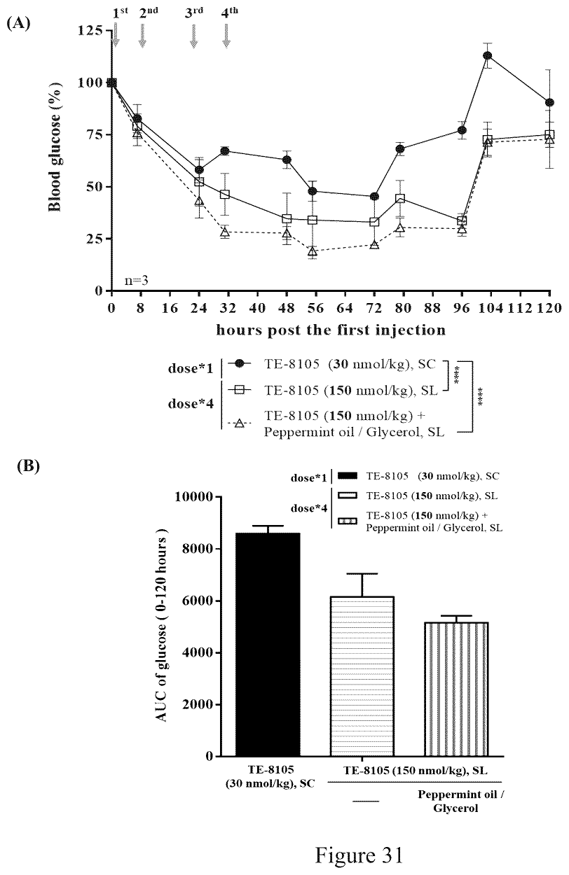

In this example, TE-8105 liquid formulation with or without the further addition of peppermint oil and glycerol were prepared in accordance with the compositions listed in Table 1. The thus prepared formulations were then administered to db/db mice either sublingually or subcutaneously twice daily at the designated dosage for two days, and blood glucose levels during the entire period of 120 hrs were measured. Results are illustrated in FIG. 31.

TABLE 1
TE-8105 liquid formulation with peppermint and glycerol

| Composition | Concentration | Remarks |
|---|---|---|
| TE-8105 | 1.4 mg/mL | Active ingredient |
| Histidine | 1.24 mg/mL | Buffer |
| Phenol | 5.5 mg/mL | Preservative |
| Mannitol | 40.0 mg/mL | Osmolarity adjustor |
| 0.05N HCl | Adjusted to pH 7-8 | |
| 0.05N NaOH | | |

Additional ingredients tested

| | | |
|---|---|---|
| Glycerol | 1.6% | Osmolyte, emollient |
| Peppermint oil | 0.1% | Moisturizer, emollient |

It was found that sublingual administration of TE-8105 liquid formulation with the addition of peppermint oil and glycerol resulted in pronounced reduction in blood glucose levels than of TE-8105 delivered by subcutaneous injection. However, whether the inclusion of peppermint oil and glycerol did possess additional statistically significant effects on blood glucose level would require further investigation.

### Example 14. Effects on Blood Glucose, Body Weight, and Food and Water Intake in db/db Mice with the Sublingual Administration of a Soft Gel of TE-8105

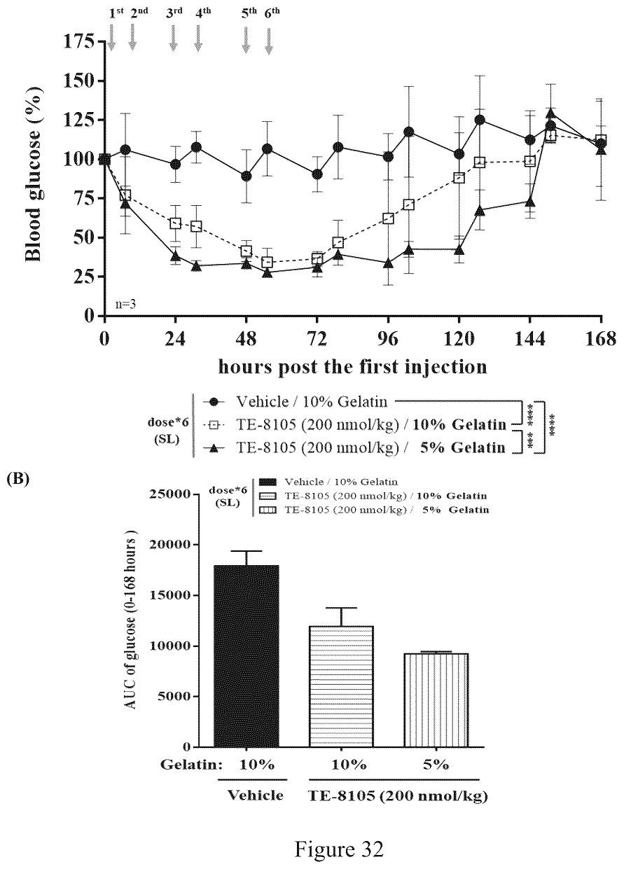

In this example, effects of TE-8105 in a soft gel form on blood glucose, body weight, and food and water intake in db/db mice were investigated. To this purpose, a disk-like soft gel of TE-8105 was prepared by mixing the TE-8105 with liquid formulation with 5% or 10% gelatin derived from cold-water fish or pig. The thus prepared TE-8105 soft gel was administered sublingually to db/db mice twice daily for 3 days, and the blood glucose, body weight, and food and water intake of the mice were measured. Results are illustrated in FIGS. 32 to 34.

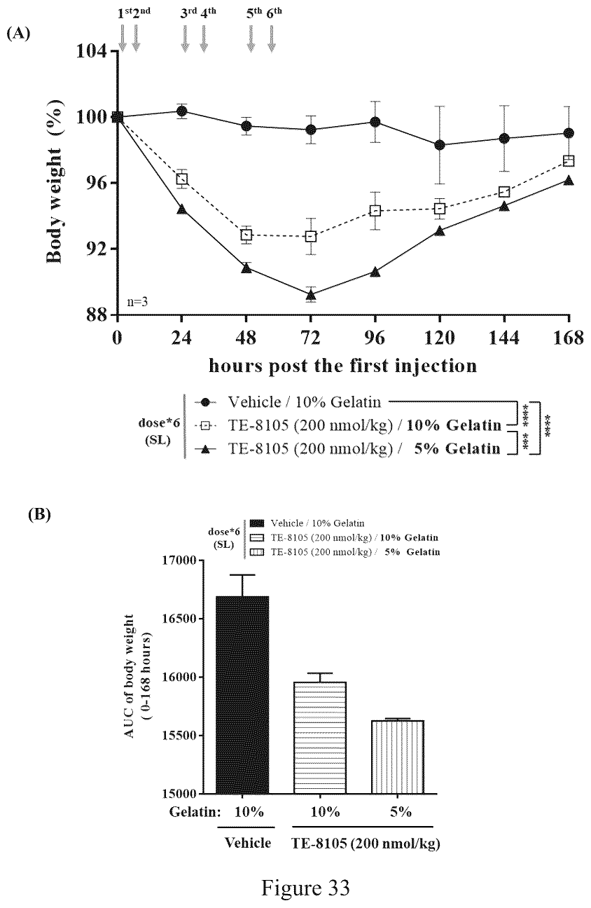

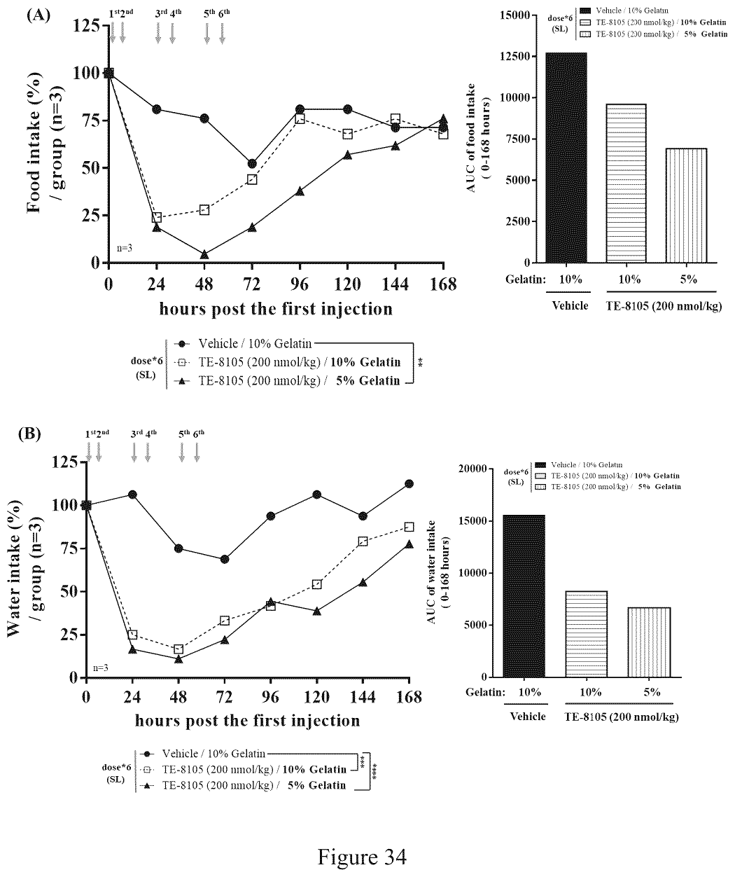

It was found that upon delivery the soft gel to the sublingual cavity of a db/db mouse, the gel would melt in 1-2 minutes. Further, the gels of TE-8105 mixed with 5% gelatin or 10% gelatin both produced significant blood glucose lowering effects to greater extents than the mock gelatin gel, which did not contain TE-8105. The data showed that TE-8105 with 5% gelatin was better than that with 10% gelatin in lowering blood glucose level (FIG. 32). In addition, the patterns on reducing body weight appeared to mirror those for glucose lowering (FIG. 33); while the pattern for the reduction of food intake mirrored to that of water intake (FIG. 34).

### Example 15. Effects on Blood Glucose, Body Weight, and Food and Water Intake in db/db Mice Upon Sublingual Administration of a Soft Gel Made by Mixing Semaglutide Liquid Formulation with Gelatin

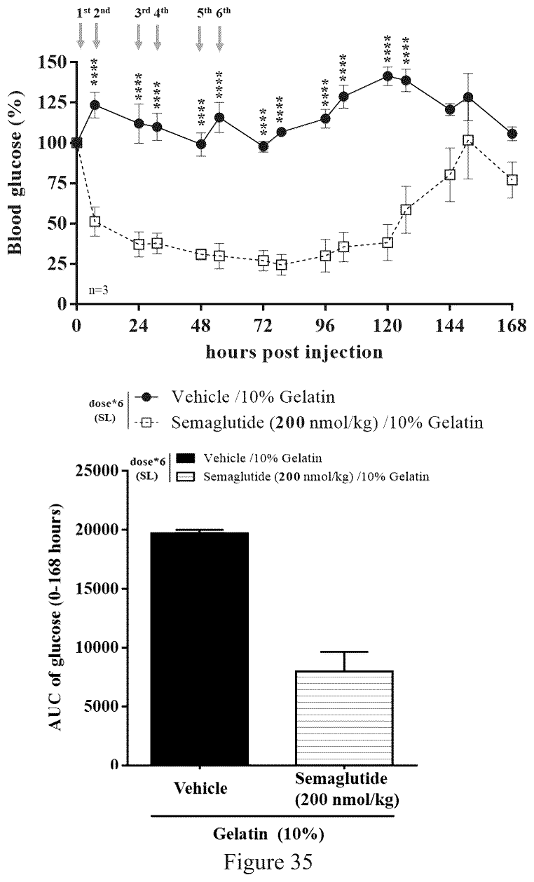

In this example, effects of semaglutide in a soft gel form on blood glucose, body weight, and food and water intake in db/db mice were investigated. The preparation of the soft gels (i.e., semaglutide liquid formulation and fish gelatin) was same as that with TE-8105 described in Example 14. The soft gel was administered sublingually twice daily for 3 days, and blood glucose, body weight, and food and water intake were measured. Results are provided in FIGS. 35 to 37.

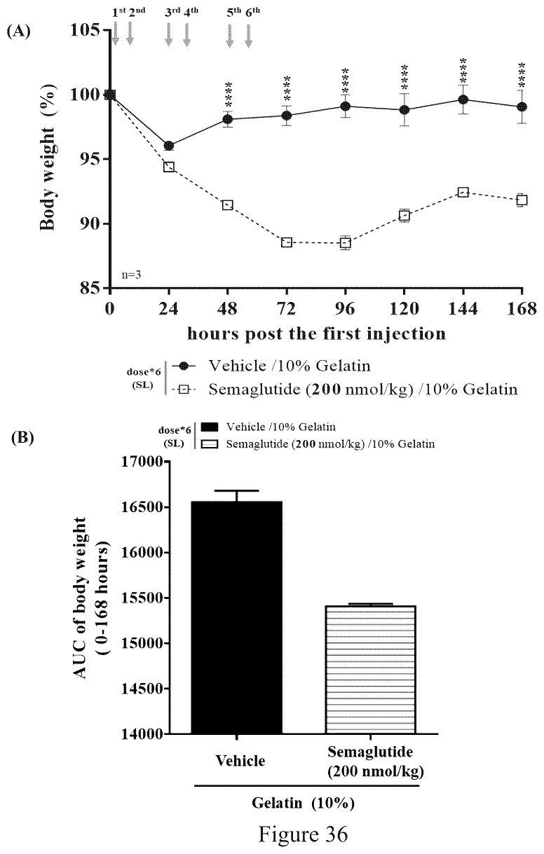

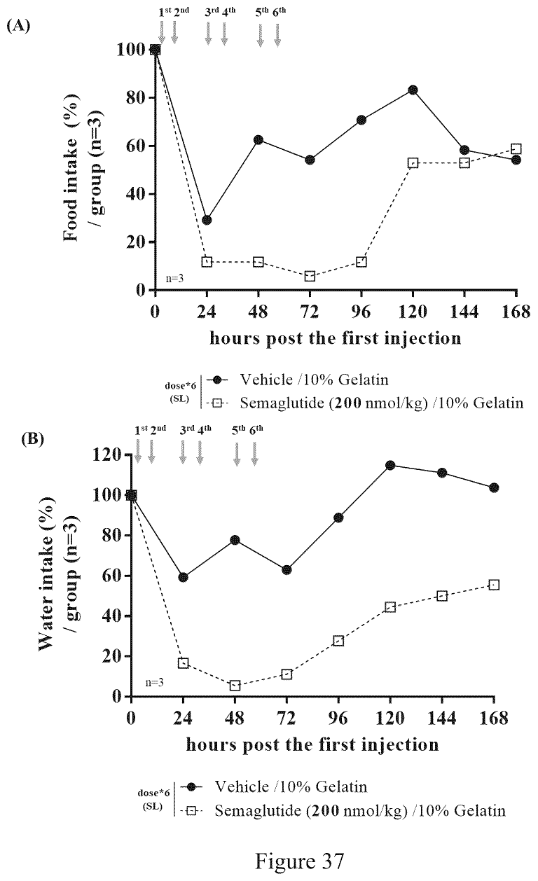

It was found that the gels of semaglutide mixed with 10% gelatin produced significant blood glucose lowering effects to a greater extent than that caused by a mock gelatin gel, which did not contain semaglutide (FIG. 35). Further, the patterns on reducing body weight appeared to mirror those for glucose lowering (FIG. 36); while the pattern for the reduction of food intake mirrored that of water intake (FIG. 37).

### Example 16. Effects on Blood Glucose, Body Weight, and Food and Water Intake in db/db Mice with Sublingual Administration of Tablets Made by Mixing TE-8105 Liquid Formulation with Crospovidone

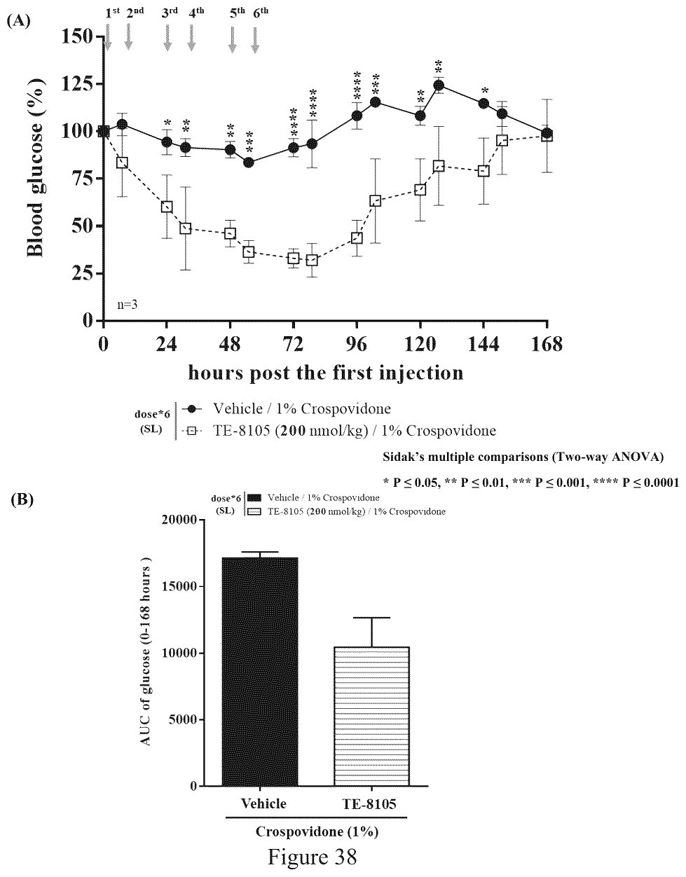

In this example, TE-8105 tablets suitable for sublingual administration were prepared. To this end, the present TE-8105 liquid formulation was mixed with crospovidone, which is a highly cross-linked modification of polyvinylpyrrolidone (PVP) and in general serves as a disintegrant in tablets. The thus produced TE-8105 tablets were administered sublingually twice daily to db/db mice for 3 days. However, since the db/db mice were anesthetized during the delivery of the tablets and their tongue and mouth were kept still, there was no saliva in the sublingual region to cause the disintegration of the tablets. Accordingly, a small amount of distilled water was placed in the sublingual cavity to help the tablets to disintegrate. Results are illustrated in FIGS. 38 to 40.

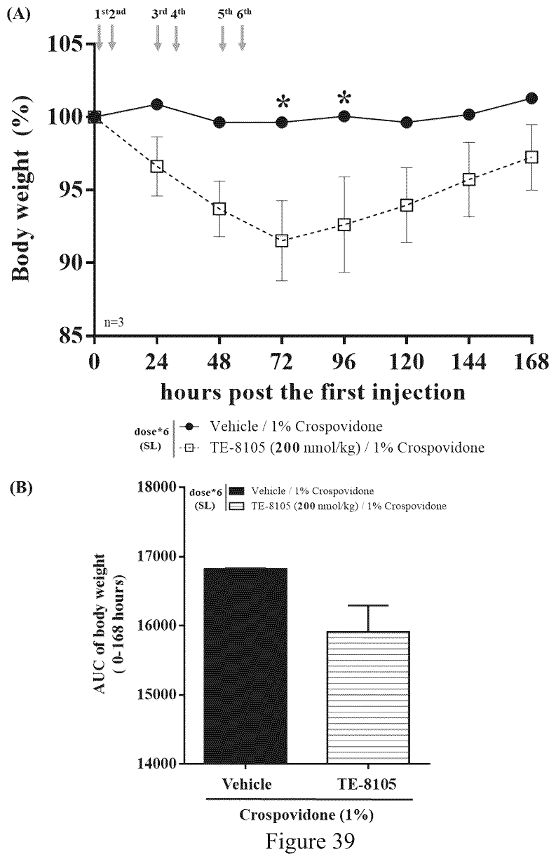

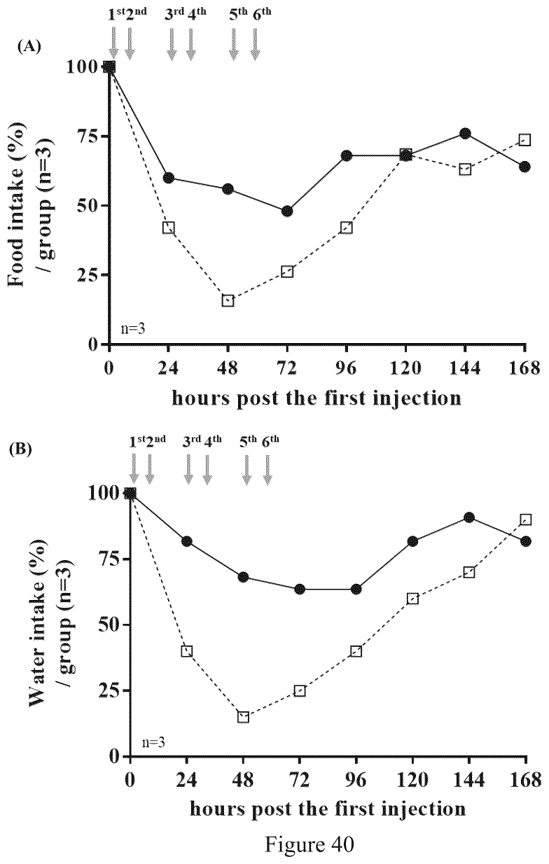

It was found that TE-8105 tablets with 1% crospovidone could reduce the high blood glucose levels to normal levels in the diabetic db/db mice (FIG. 38). Further, the TE-8105 tablets could effectively reduce the body weight, as well as food and water intake of the obese db/db mice (FIGS. 39 and 40).

### Example 17. Effects on Blood Glucose, Body Weight, and Food and Water Intake in db/db Mice Upon the Sublingual Administration of Tablets Made by Mixing Semaglutide Liquid Formulation with Crospovidone

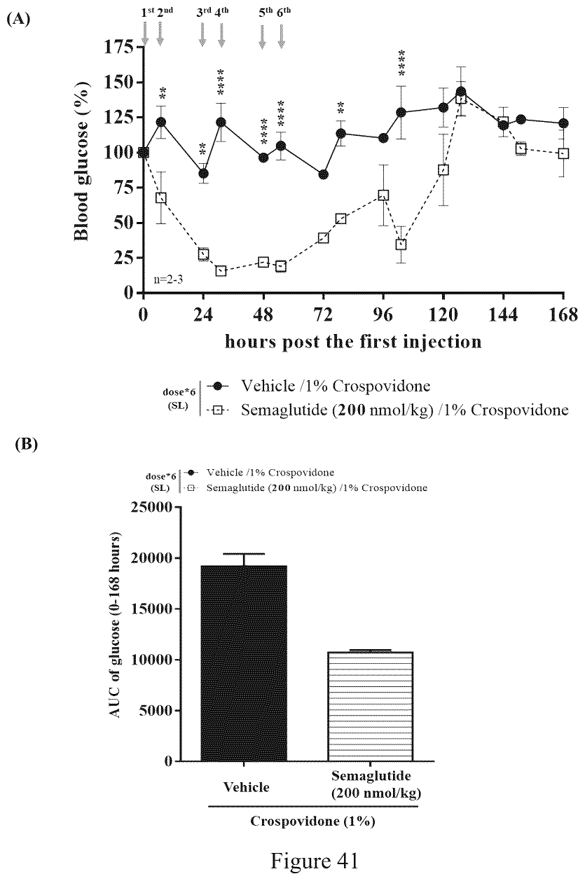

In this example, semaglutide tablets (i.e., mixing semaglutide liquid formulation with 1% crospovidone) were prepared and administered in accordance with similar procedures described in Example 16, and their effects on blood glucose control were monitored. Results are illustrated in FIGS. 41 to 43.

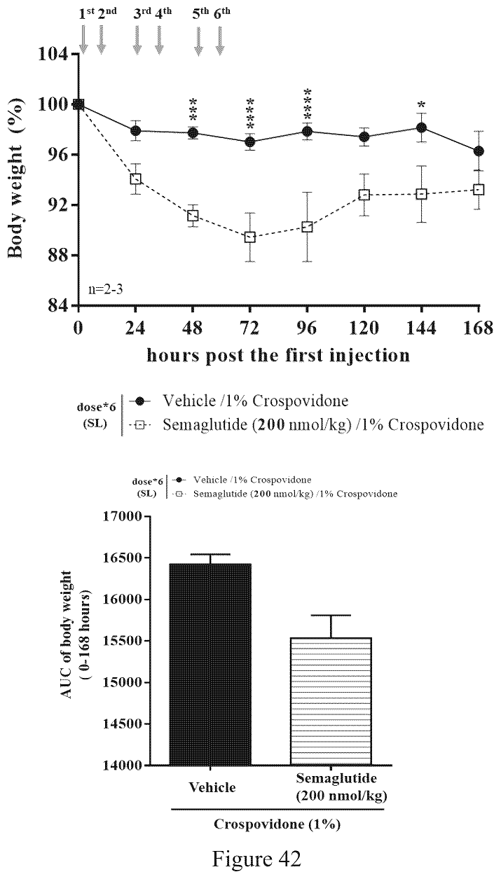

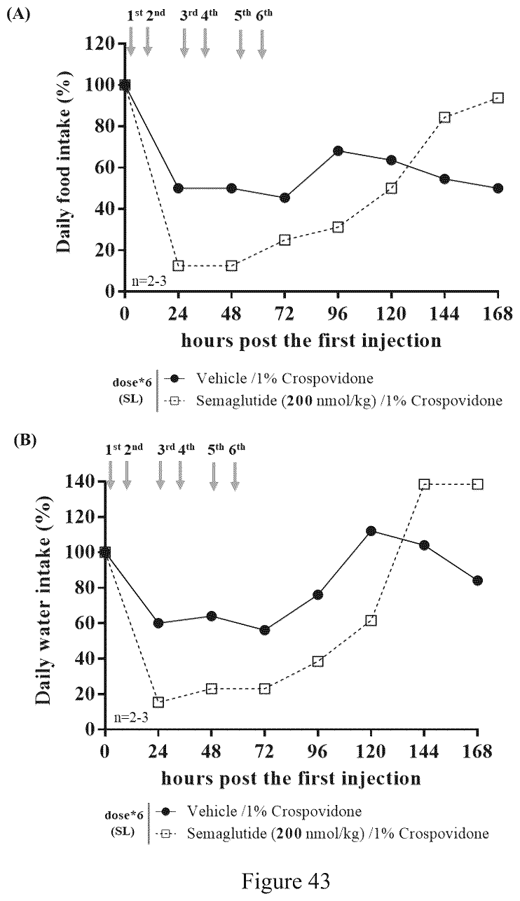

It was found that semaglutide tablets could effectively reduce high blood glucose to normal levels in the db/db mice (FIG. 41). Further, semaglutide tablets could also effectively reduce body weight (FIG. 42), and food and water intake (FIG. 43).

### Example 18. Effects on Blood Glucose, Body Weight, and Food and Water Intake in db/db Mice Upon the Sublingual Administration of TE-8105 Peppermint Tablets

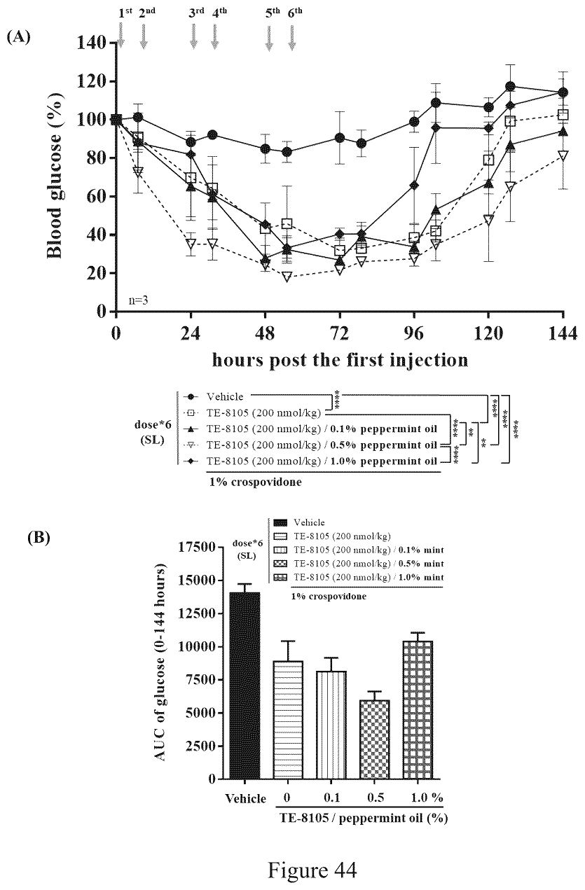

In this example, TE-8105 peppermint tablets were prepared in accordance with procedures described in Example 16 with additional 0.1%, 0.5%, or 1.0% peppermint oil added therein to mix with TE-8105 liquid formulation and 1% crospovidone. Effects of the TE-8105 peppermint tablets on blood glucose control were monitored in db/db mice. Results are illustrated in FIGS. 44 to 47.

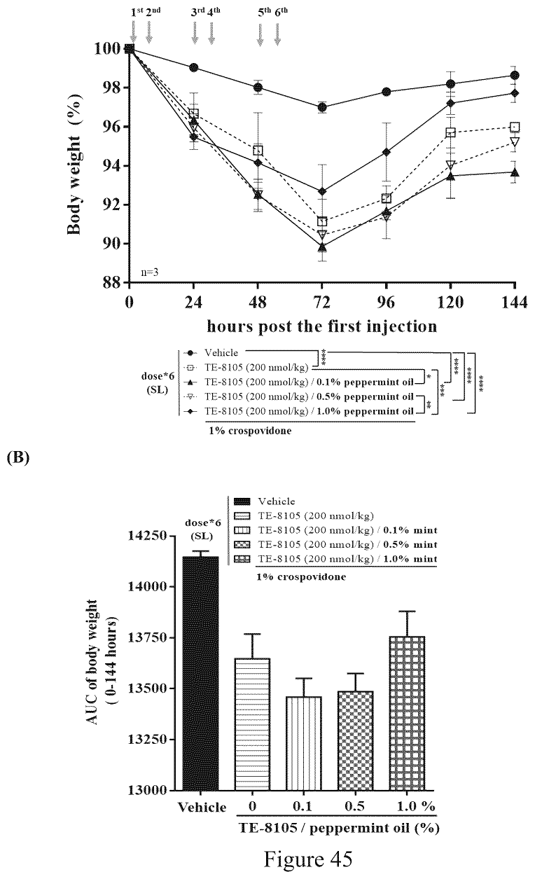

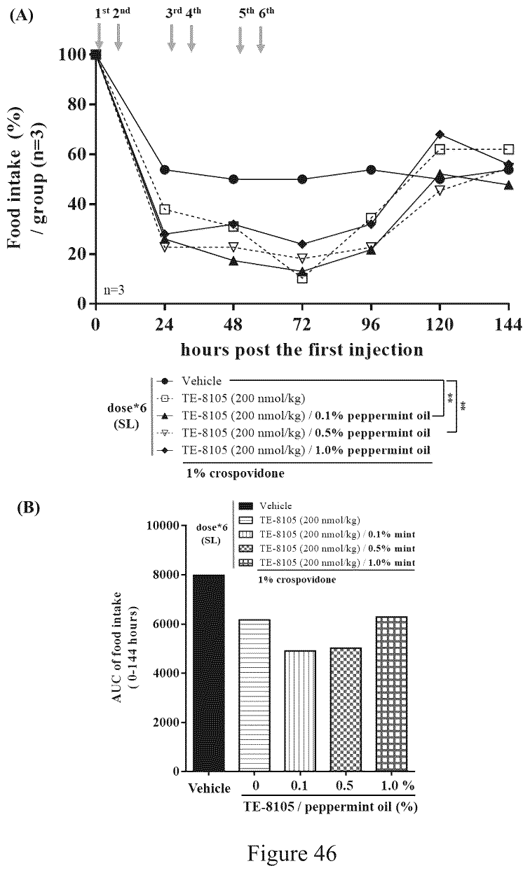

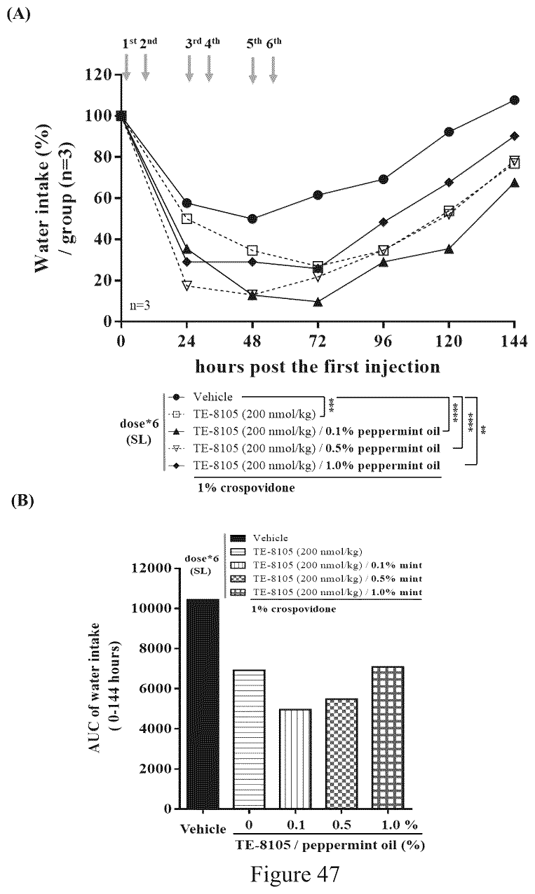

It was found that the TE-8105 tablets without peppermint and TE-8105 tablets with 0.1%, 0.5%, or 1.0% peppermint oil all could effectively reduce blood glucose levels, with those containing 0.5% peppermint being the most effective (FIG. 44). In addition, TE-8105 tablets without peppermint and TE-8105 tablets with 0.1%, 0.5%, or 1.0% peppermint oil all could effectively reduce body weight, with those containing 0.1% and 0.5% exhibited better results (FIG. 45). As to food intake, the TE-8105 tablets with 0.1%, and 0.5% peppermint oil could significantly reduce food intake in the db/db mice; the other TE-8105 tablets also displayed a trend of reducing food intake (FIG. 46). For water intake, all TE-8105 tablets reduced water intake in db/db mice, with those containing 0.1% or 0.5% peppermint oil being the most effective (FIG. 47).

### Example 19. Estimation of Dosing Protocols for TE-8105, Semaglutide, and Tirzepatide in Human Subjects Based on the Bioavailability in Mouse Models

Results from Examples 2 to 18 confirmed that the delivered fluid having a GLP-1 RA-P therein could be retained in the sublingual area of db/db mice, while the mice were kept in anesthetized condition for about 5-7 minutes. There has not been a published procedure to demonstrate that the liquid formulation of a drug applied to the sublingual region may be retained entirely in a local area for substantial time. It is understood from literature survey that a human subject can hold a small volume of liquid in the sublingual cavity for a short time, in the range of 5-10 minutes, without pushing the liquid out of the sublingual area due to mouth or tongue movement. Further, it was found from the published reference that 1.5 to 2.0 ml is the maximal adequate volume to deliver to the sublingual region of a human subject. In the present disclosure, the time for optimal sublingual absorption of TE-8105 by the db/db mice was identified to be 5 to 10 minutes (Example 7).

In our experiments, we prepared the stock solution of TE-8105 and tirzepatide in liquid formulation at 5 mg/ml from powder. These stock solutions would then be diluted with the liquid formulation buffer for other concentrations. We also employed the drug product of TE-8105, which was at 5 mg/ml in the formulation liquid, provided by the contracted CDMO service. It was found that the optimal concentration for facilitating transmucosal transport through the sublingual region is at 3-5 mg/ml concentration (Example 6). The effect of concentration on the efficiency of transmucosal absorption is believed to be related to molecular interactions between the GLP-1 RA-Ps and the molecules on the surface of mucosal epithelial cells. These findings align well with the solubility of TE-8105 (5 mg/ml) and semaglutide (3.2 mg/ml) in aqueous buffer. Tirzepatide, due to its terminal nine amino acid residues, has a very high solubility in aqueous buffer, 30 mg/ml. However, the transport across the sublingual mucosal epithelium is probably limited to the optimal concentration of 3-5 mg/ml.

The solubility of TE-8105 is based on the drug product in liquid formulation (5 mg in 1 mL solution) employed in Phase I/IIA clinical trials. The solubility of semaglutide in the liquid formulation for subcutaneous injection is based on the known highest dosage form (0.24 mg in 0.75 mL solution). Therefore, for TE-8105, 2 ml liquid formulation can contain 10 mg; for semaglutide, 2 ml liquid formulation can contain 6.4 mg; for tirzepatide, we would rationalize that 2 ml of liquid formulation can also contain 10 mg.

In Table 2, the amounts and frequency of TE-8105, semaglutide, and tirzepatide required for sublingual delivery are based on their maintenance dosing for subcutaneous injection and the projected range of bioavailability in human subjects. Conditions indicated in bold letters represent feasible delivery options, assuming the projected bioavailability is achieved. For each GLP-1 RA-P under a given projected bioavailability, only the dosing condition requiring the least frequent administration is listed. For instance, if 10 mg once weekly is feasible, it is included, while the more frequent 5 mg twice weekly or 1.4 mg daily options are not listed; another example, if 4.6 mg once per two days is feasible, it is listed, while 2.3 mg per day and 1.2 mg twice per day are not listed.

The dosing frequency symbols used are:
2QD: twice per day
QD: once per day
Q2D: once every two days
2QW: twice per week
QW: once per week
Q2W: once every two weeks”

TABLE 2
The amounts and dosing frequency for the sublingual administration
of TE-8105, semaglutide, and tirzepatide.

| | Amounts and frequency of GLP-1 RA-Ps | | |
|---|---|---|---|
| Maximal dosing for | required for sublingual delivery | | |
| subcutaneous | Projected bioavailability | | |
| administration | 2% | 5% | 10% |
| 15 mg QW | 750 mg QW | 300 mg QW | 150 mg QW |
| (tirzepatide) | 107 mg QD | 43 mg QD | 21 mg QD |
| | | | 10 mg 2QD |
| 2.4 mg QW | 120 mg QW | 48 mg QW | 24 mg QW |
| (semaglutide) | 17 mg QD | 6.8 mg QD | 6.8 mg Q2D |
| | | 3.4 mg 2QD | 3.4 mg QD |
| 2 mg Q2W | 100 mg Q2W | 40 mg Q2W | 20 mg Q2W |
| (TE-8105) | 50 mg QW | 20 mg QW | 10 mg QW |
| | 7.2 mg QD | 10 mg 2QW | |

Note:
Those options in bold letters are feasible under the projection of bioavailability. The projected bioavailability is the percentage achieved relative to that of subcutaneous delivery.

In sum, data of the present disclosure confirms that the present sublingual formulations, upon administrating to a subject in need, could achieve an unexpected high level of bioavailability of GLP-1 RA-P that is about 7-10%, or 2-10% adjusted for potential variations among human subjects, of that achieved by subcutaneous injection, and results in a reduction in blood glucose level, body weight and food and water intake of the subject, thus are useful for treating obesity, type II diabetes or disorders related thereto.

It will be understood that the above description of embodiments is given by way of examples only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A pharmaceutical formulation for treating obesity, Type II diabetes or a disorder related thereto comprising:
5-10 mg of a glucagon-like peptide-1 receptor agonist peptide (GLP-1 RA-P) dissolved in 1.5-2.0 mL of a buffer solution;
wherein,
the GLP-1 RA-P is selected from the group consisting of TE-8105, semaglutide, and tirzepatide, in which TE-8105 has the structure as depicted in FIG. 1;
the pharmaceutical formulation has a pH value of 5.5 to 7.5 and is suitable for sublingual administration; and
the pharmaceutical formulation results in 2-10% bioavailability of the GLP-1 RA-P as compared to that achieved by subcutaneous injection upon being administered to a subject.

2. The pharmaceutical formulation of claim 1, wherein the pharmaceutical formulation is in the form of a liquid, a tablet, or a soft gel.

3. The pharmaceutical formulation of claim 2, wherein the pharmaceutical formulation is in the form of the liquid and comprises 5-10 mg TE-8105 dissolved in 1.5-2.0 mL of a buffer solution.

4. The pharmaceutical formulation of claim 2, wherein the pharmaceutical formulation is in the form of the liquid and comprises 5-6 mg semaglutide dissolved in 1.5-2.0 mL of a buffer solution.

5. The pharmaceutical formulation of claim 2, wherein the pharmaceutical formulation is in the form of the liquid and comprises 5-10 mg tirzepatide dissolved in 1.5-2.0 mL of a buffer solution.

6. The pharmaceutical formulation of claim 2, further comprising 0.1% (w/v) peppermint oil.

7. The pharmaceutical formulation of claim 2, further comprising 5-10% gelatin, and the formulation is in the form of the soft gel.

8. The pharmaceutical formulation of claim 2, further comprising 1-10% crospovidone, and the formulation is in the form of the tablet.

9. The pharmaceutical formulation of claim 1, wherein the disorder related to obesity or Type II diabetes is overweight, fatty liver disease, nonalcoholic steatohepatitis, diabetic cardiomyopathy, or atherosclerotic cardiovascular disease.

10. A method for treating obesity, Type II diabetes or a disorder related thereto in a subject comprising:
sublingually administering an effective amount of a pharmaceutical formulation comprising a glucagon-like peptide-1 receptor agonist peptide (GLP-1 RA-P) to the subject, in which the pharmaceutical formulation is kept under the tongue of the subject for 5 to 10 minutes;
wherein,
the GLP-1 RA-P is selected from the group consisting of TE-8105, semaglutide and tirzepatide, in which TE-8105 has the structure as depicted in FIG. 1B;
the pharmaceutical formulation has a pH value of 5.5 to 7;
the pharmaceutical formulation is administered once daily, twice daily, once every two days, once weekly, or twice weekly; and
the sublingual administration of the pharmaceutical formulation achieves 2-10% bioavailability of the GLP-1 RA-P as compared to that achieved by subcutaneous injection.

11. The method of claim 10, wherein the pharmaceutical formulation is in the form of a liquid, a tablet, or a soft gel.

12. The method of claim 11, wherein
the pharmaceutical formulation is in the form of the liquid and comprises 5-10 mg TE-8105 dissolved in 1.5-2.0 mL of a buffer solution; and
the pharmaceutical formulation is administered once daily, twice daily, once every two days, once weekly, or twice weekly.

13. The method of claim 11, wherein
the pharmaceutical formulation is in the form of the liquid and comprises 5-6 mg semaglutide dissolved in 1.5-2.0 mL of a buffer solution; and
the pharmaceutical formulation is administered once daily or once every two days.

14. The method of claim 11, wherein
the pharmaceutical formulation is in the form of the liquid and comprises 5-10 mg tirzepatide dissolved in 1.5-2.0 mL of a buffer solution, and
the pharmaceutical formulation is administered twice daily.

15. The method of claim 11, wherein the pharmaceutical formulation further comprises 0.01% (w/v) peppermint oil.

16. The method of claim 11, wherein the pharmaceutical formulation further comprises 5-10% (w/v) gelatin, and is in the form of the soft gel.

17. The method of claim 11, wherein the pharmaceutical formulation further comprises 1-10% (w/v) crospovidone, and is in the form of the tablet.

18. The method of claim 10, wherein the disorder related to obesity or Type II diabetes is overweight, fatty liver disease, nonalcoholic steatohepatitis, diabetic cardiomyopathy, or atherosclerotic cardiovascular disease.

19. The method of claim 10, wherein the subject is a human.
